(19) United States
(12) Patent Application Publication
(10) Pub. No.: US 2026/0317417 A1
(43) Pub. Date: Oct. 15, 2026

(54) METHODS FOR TREATING IL-6 MEDIATED INFLAMMATION WITHOUT IMMUNOSUPPRESSION

(71) Applicant: Novo Nordisk A/S, Bagsvaerd (DK)

(72) Inventors: Madhav N. Devalaraja, Acton, MA (US); Michael H. Davidson, Highland Park, IL (US); Rahul Kakkar, Weston, MA (US)

(21) Appl. No.: 19/650,529

(22) Filed: Apr. 17, 2026

Related U.S. Application Data
(63) Continuation of application No. 17/991,880, filed on Nov. 22, 2022.
Application No. 17/991,880 is a continuation of application No. 17/708,660, filed on Mar. 30, 2022.
Application No. 17/708,660 is a continuation of application No. 16/396,378, filed on Apr. 26, 2019, now Pat. No. 11,384,143.
Application No. 16/396,378 is a continuation of application No. 16/240,670, filed on Jan. 4, 2019, now abandoned.
(60) Provisional application No. 62/614,134, filed on Jan. 5, 2018.

(51) Int. Cl. C07K 16/24; A61K 39/00; A61K 39/395; A61P 13/12; A61P 19/02; A61P 37/02

(52) U.S. Cl. CPC C07K 16/248; A61K 39/3955; A61P 13/12; A61P 19/02; A61P 37/02; A61K 2039/505; A61K 2039/545; A61K 2039/55; C07K 2317/52; C07K 2317/76

(57) ABSTRACT

The disclosure provides methods of treating inflammation without inducing immune suppression. The method comprises administering a therapeutically effective amount of an IL-6 antagonist at a dose sufficient to reduce inflammation without causing immune suppression.

### CROSS REFERENCE TO RELATED APPLICATIONS

This application is a continuation of co-pending U.S. application Ser. No. 17/991,880, filed Nov. 22, 2022, which is a continuation of U.S. application Ser. No. 17/708,660, filed Mar. 30, 2022, which is a continuation of U.S. application Ser. No. 16/396,378, filed Apr. 26, 2019 (now U.S. Pat. No. 11,384,143, issued Jul. 12, 2022), which is a continuation of U.S. application Ser. No. 16/240,670, filed Jan. 4, 2019 (now abandoned), which claims priority to U.S. Provisional Application No. 62/614,134, filed Jan. 5, 2018, each of which is hereby incorporated in its entirety by reference.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on Apr. 7, 2026, is named 200110US06_CON_SequenceListing.xml and is 20,274 bytes in size.

### BACKGROUND

Chronic inflammation is a characteristic of many diseases, including both the classical rheumatic disorders such as rheumatoid arthritis, juvenile idiopathic arthritis, psoriatic arthritis, and inflammatory bowel disease, as well as other systemic diseases that are increasingly understood to be associated with chronic inflammation, such as cardiovascular disease, renal disease, neuroinflammatory diseases, anemias, cancer and aging.

The pro-inflammatory cytokine, IL-6, often plays a critical role in chronic inflammation through activation of the JAK-STAT signaling pathway, and IL-6 inhibitors have been developed to treat certain inflammatory disorders in which IL-6 has been shown to contribute significantly to disease etiology. The anti-IL-6 receptor antibody, tocilizumab (ACTEMRA), has been approved for treatment of rheumatoid arthritis, giant cell arteritis, polyarticular juvenile idiopathic arthritis, systemic juvenile idiopathic arthritis, and iatrogenic cytokine release syndrome. The anti-IL-6 receptor antibody, sarilumab (KEVZARA), has been approved to treat adult patients with moderately to severely active rheumatoid arthritis.

Although inhibition of IL-6 can be effective, treatment of chronic inflammation with IL-6 inhibitors using current dose regimens often leads to immune suppression. Immunosuppression can result in increased susceptibility to pathogens such as bacteria, fungi, and viruses. The FDA-approved product label for ACTEMRA warns of the risk of serious infections leading to hospitalization or death, including tuberculosis, bacterial, invasive fungal, viral, and other opportunistic infection; the KEVZARA label warns of serious infections leading to hospitalization or death including bacterial, viral, invasive fungal, and other opportunistic infections.

There is, therefore, a need for new methods for treating IL-6 mediated inflammation that do not lead to immune suppression.

### SUMMARY

We have demonstrated that IL-6 antagonists can be administered at a dose, on a schedule, and for a period sufficient to reduce inflammation without causing immune suppression.

Accordingly, in a first aspect, methods for treating IL-6-mediated inflammation in a patient are provided. The methods comprise: administering an IL-6 antagonist to a patient with IL-6-mediated inflammation at a dose that is sufficient to reduce inflammation without causing immune suppression.

In some embodiments, the patient has an elevated pre-treatment C-reactive protein (CRP) level. In some embodiments, the pre-treatment CRP level of the patient is at least 2 mg/L. In some embodiments, the pre-treatment CRP level of the patient is at least 4 mg/L. In some embodiments, the pre-treatment CRP level of the patient is at least 6 mg/L. In some embodiments, the pre-treatment CRP level of the patient is at least 10 mg/L.

In some embodiments, the patient has an elevated pre-treatment serum IL-6 level. In some embodiments, the pre-treatment serum IL-6 level of the patient is at least 4 pg/mL. In some embodiments, the pre-treatment serum IL-6 level of the patient is at least 4 pg/mL. In some embodiments, the pre-treatment serum IL-6 level of the patient is at least 5 pg/mL. In some embodiments, the pre-treatment serum IL-6 level of the patient is at least 10 pg/mL.

In some embodiments, the inflammation is measured by the level of C-reactive protein (CRP). In some embodiments, the post-treatment CRP level is no more than 2 mg/L. In some embodiments, the post-treatment CRP level is no more than 1 mg/L. In some embodiments, the CRP level is decreased by at least 50% as compared to pre-treatment levels. In some embodiments, the CRP level is decreased by at least 70% as compared to pre-treatment levels. In some embodiments, the CRP level is decreased by at least 80% as compared to pre-treatment levels. In some embodiments, the CRP level is decreased by at least 90% as compared to pre-treatment levels.

In some embodiments, the immune suppression is measured by absolute neutrophil count (ANC). In some embodiments, the post-treatment ANC is at least 500 cells/μL. In some embodiments, the post-treatment ANC is at least 1000 cells/μL. In some embodiments, the post-treatment ANC is at least 1500 cells/μL. In some embodiments, the post-treatment ANC is at least 2000 cells/μL. In some embodiments, the ANC is decreased by no more than 2000 cells/μL as compared to pre-treatment levels. In some embodiments, the ANC is decreased by no more than 1500 cells/μL as compared to pre-treatment levels. In some embodiments, the ANC is decreased by no more than 1000 cells/μL as compared to pre-treatment levels. In some embodiments, the ANC is decreased by no more than 500 cells/μL as compared to pre-treatment levels. In some embodiments, the ANC is decreased by no more than 50% as compared to pre-treatment levels. In some embodiments, the ANC is decreased by no more than 40% as compared to pre-treatment levels. In some embodiments, the ANC is decreased by no more than 30% as compared to pre-treatment levels. In some embodiments, the ANC is decreased by no more than 20% as compared to pre-treatment levels. In some embodiments, the ANC is decreased by no more than 10% as compared to pre-treatment levels. In some embodiments, the ANC is not decreased as compared to pre-treatment levels.

In some embodiments, the IL-6 antagonist is administered at a monthly equivalent dose that is no more than 30% of the monthly equivalent dose for treating rheumatoid arthritis with the same IL-6 antagonist. In some embodiments, the IL-6 antagonist is administered at a monthly equivalent dose that is no more than 20% of the monthly equivalent dose for treating rheumatoid arthritis with the same IL-6 antagonist. In some embodiments, the IL-6 antagonist is administered at a monthly equivalent dose that is no more than 10% of the monthly equivalent dose for treating rheumatoid arthritis with the same IL-6 antagonist. In some embodiments, the IL-6 antagonist is administered at a monthly equivalent dose that is about 25% of a monthly equivalent dose for treating rheumatoid arthritis with the same IL-6 antagonist. In some embodiments, the IL-6 antagonist is administered at a monthly equivalent dose that is about 20% of a monthly equivalent dose for treating rheumatoid arthritis with the same IL-6 antagonist. In some embodiments, the IL-6 antagonist is administered at a monthly equivalent dose that is about 15% of a monthly equivalent dose for treating rheumatoid arthritis with the same IL-6 antagonist. In some embodiments, the IL-6 antagonist is administered at a monthly equivalent dose that is about 10% of a monthly equivalent dose for treating rheumatoid arthritis with the same IL-6 antagonist. In some embodiments, the IL-6 antagonist is administered at a monthly equivalent dose that is about 5% of a monthly equivalent dose for treating rheumatoid arthritis with the same IL-6 antagonist.

In some embodiments, the IL-6 antagonist is an anti-IL-6 antibody.

In some embodiments, the anti-IL-6 antibody is COR-001. In some embodiments, COR-001 is administered intravenously at a monthly equivalent dose of 2-40 mg. In some embodiments, COR-001 is administered intravenously at a monthly equivalent dose of about 2 mg. In some embodiments, COR-001 is administered intravenously at a monthly equivalent dose of about 4 mg. In some embodiments, COR-001 is administered intravenously at a monthly equivalent dose of about 6 mg. In some embodiments, COR-001 is administered intravenously at a monthly equivalent dose of about 10 mg. In some embodiments, COR-001 is administered intravenously at a monthly equivalent dose of about 20 mg. In some embodiments, COR-001 is administered intravenously at a monthly equivalent dose of about 40 mg. In some embodiments, COR-001 is administered subcutaneously at a monthly equivalent dose of 3-70 mg. In some embodiments, COR-001 is administered subcutaneously at a monthly equivalent dose of about 3 mg. In some embodiments, COR-001 is administered subcutaneously at a monthly equivalent dose of about 7 mg. In some embodiments, COR-001 is administered subcutaneously at a monthly equivalent dose of about 10 mg. In some embodiments, COR-001 is administered subcutaneously at a monthly equivalent dose of about 17 mg. In some embodiments, COR-001 is administered subcutaneously at a monthly equivalent dose of about 35 mg. In some embodiments, COR-001 is administered subcutaneously at a monthly equivalent dose of about 70 mg.

In some embodiments, the anti-IL-6 antibody is siltuximab. In some embodiments, siltuximab is administered intravenously at a monthly equivalent dose of 50-500 mg. In some embodiments, siltuximab is administered intravenously at a monthly equivalent dose of about 50 mg. In some embodiments, siltuximab is administered intravenously at a monthly equivalent dose of about 100 mg. In some embodiments, siltuximab is administered intravenously at a monthly equivalent dose of about 150 mg. In some embodiments, siltuximab is administered intravenously at a monthly equivalent dose of about 200 mg. In some embodiments, siltuximab is administered intravenously at a monthly equivalent dose of about 300 mg. In some embodiments, siltuximab is administered intravenously at a monthly equivalent dose of about 500 mg. In some embodiments, siltuximab is administered subcutaneously at a monthly equivalent dose of 80-800 mg. In some embodiments, siltuximab is administered subcutaneously at a monthly equivalent dose of about 80 mg. In some embodiments, siltuximab is administered subcutaneously at a monthly equivalent dose of about 160 mg. In some embodiments, siltuximab is administered subcutaneously at a monthly equivalent dose of about 240 mg. In some embodiments, siltuximab is administered subcutaneously at a monthly equivalent dose of about 320 mg. In some embodiments, siltuximab is administered subcutaneously at a monthly equivalent dose of about 480 mg. In some embodiments, siltuximab is administered subcutaneously at a monthly equivalent dose of about 800 mg.

In some embodiments, the anti-IL-6 antibody is gerilimzumab. In some embodiments, gerilimzumab is administered intravenously at a monthly equivalent dose of 0.075-1.8 mg. In some embodiments, gerilimzumab is administered intravenously at a monthly equivalent dose of about 0.075 mg. In some embodiments, gerilimzumab is administered intravenously at a monthly equivalent dose of about 0.12 mg. In some embodiments, gerilimzumab is administered intravenously at a monthly equivalent dose of about 0.3 mg. In some embodiments, gerilimzumab is administered intravenously at a monthly equivalent dose of about 0.6 mg. In some embodiments, gerilimzumab is administered intravenously at a monthly equivalent dose of about 0.9 mg. In some embodiments, gerilimzumab is administered intravenously at a monthly equivalent dose of about 1.8 mg. In some embodiments, gerilimzumab is administered subcutaneously at a monthly equivalent dose of 0.125-3 mg. In some embodiments, gerilimzumab is administered subcutaneously at a monthly equivalent dose of about 0.125 mg. In some embodiments, gerilimzumab is administered subcutaneously at a monthly equivalent dose of about 0.2 mg. In some embodiments, gerilimzumab is administered subcutaneously at a monthly equivalent dose of about 0.5 mg. In some embodiments, gerilimzumab is administered subcutaneously at a monthly equivalent dose of about 1 mg. In some embodiments, gerilimzumab is administered subcutaneously at a monthly equivalent dose of about 1.5 mg. In some embodiments, gerilimzumab is administered subcutaneously at a monthly equivalent dose of about 3 mg.

In some embodiments, the anti-IL-6 antibody is sirukumab. In some embodiments, sirukumab is administered intravenously at a monthly equivalent dose of 1.5-60 mg. In some embodiments, sirukumab is administered intravenously at a monthly equivalent dose of about 1.5 mg. In some embodiments, sirukumab is administered intravenously at a monthly equivalent dose of about 3 mg. In some embodiments, sirukumab is administered intravenously at a monthly equivalent dose of about 6 mg. In some embodiments, sirukumab is administered intravenously at a monthly equivalent dose of about 12 mg. In some embodiments, sirukumab is administered intravenously at a monthly equivalent dose of about 36 mg. In some embodiments, sirukumab is administered intravenously at a monthly equivalent dose of about 60 mg. In some embodiments, sirukumab is administered subcutaneously at a monthly equivalent dose of 2.5-100 mg. In some embodiments, sirukumab is administered subcutaneously at a monthly equivalent dose of about 2.5 mg. In some embodiments, sirukumab is administered subcutaneously at a monthly equivalent dose of about 5 mg. In some embodiments, sirukumab is administered subcutaneously at a monthly equivalent dose of about 10 mg. In some embodiments, sirukumab is administered subcutaneously at a monthly equivalent dose of about 20 mg. In some embodiments, sirukumab is administered subcutaneously at a monthly equivalent dose of about 60 mg. In some embodiments, sirukumab is administered subcutaneously at a monthly equivalent dose of about 100 mg.

In some embodiments, the anti-IL-6 antibody is clazakizumab. In some embodiments, clazakizumab is administered intravenously at a monthly equivalent dose of 3-60 mg. In some embodiments, clazakizumab is administered intravenously at a monthly equivalent dose of about 3 mg. In some embodiments, clazakizumab is administered intravenously at a monthly equivalent dose of about 6 mg. In some embodiments, clazakizumab is administered intravenously at a monthly equivalent dose of about 12 mg. In some embodiments, clazakizumab is administered intravenously at a monthly equivalent dose of about 24 mg. In some embodiments, clazakizumab is administered intravenously at a monthly equivalent dose of about 36 mg. In some embodiments, clazakizumab is administered intravenously at a monthly equivalent dose of about 60 mg. In some embodiments, clazakizumab is administered subcutaneously at a monthly equivalent dose of 5-100 mg. In some embodiments, clazakizumab is administered subcutaneously at a monthly equivalent dose of about 5 mg. In some embodiments, clazakizumab is administered subcutaneously at a monthly equivalent dose of about 10 mg. In some embodiments, clazakizumab is administered subcutaneously at a monthly equivalent dose of about 20 mg. In some embodiments, clazakizumab is administered subcutaneously at a monthly equivalent dose of about 40 mg. In some embodiments, clazakizumab is administered subcutaneously at a monthly equivalent dose of about 60 mg. In some embodiments, clazakizumab is administered subcutaneously at a monthly equivalent dose of about 100 mg.

In some embodiments, the anti-IL-6 antibody is olokizumab. In some embodiments, olokizumab is administered intravenously at a monthly equivalent dose of 1.8-60 mg. In some embodiments, olokizumab is administered intravenously at a monthly equivalent dose of about 1.8 mg. In some embodiments, olokizumab is administered intravenously at a monthly equivalent dose of about 3.6 mg. In some embodiments, olokizumab is administered intravenously at a monthly equivalent dose of about 9 mg. In some embodiments, olokizumab is administered intravenously at a monthly equivalent dose of about 18 mg. In some embodiments, olokizumab is administered intravenously at a monthly equivalent dose of about 45 mg. In some embodiments, olokizumab is administered intravenously at a monthly equivalent dose of about 60 mg. In some embodiments, olokizumab is administered subcutaneously at a monthly equivalent dose of 3-100 mg. In some embodiments, olokizumab is administered subcutaneously at a monthly equivalent dose of about 3 mg. In some embodiments, olokizumab is administered subcutaneously at a monthly equivalent dose of about 6 mg. In some embodiments, olokizumab is administered subcutaneously at a monthly equivalent dose of about 15 mg. In some embodiments, olokizumab is administered subcutaneously at a monthly equivalent dose of about 30 mg. In some embodiments, olokizumab is administered subcutaneously at a monthly equivalent dose of about 72 mg. In some embodiments, olokizumab is administered subcutaneously at a monthly equivalent dose of about 100 mg.

In some embodiments, the anti-IL-6 antibody is VX30 (VOP-R003; Vaccinex). In some embodiments, VX30 (VOP-R003; Vaccinex) is administered intravenously. In some embodiments, VX30 (VOP-R003; Vaccinex) is administered subcutaneously.

In some embodiments, the anti-IL-6 antibody is EB-007 (EBI-029; Eleven Bio). In some embodiments, EB-007 (EBI-029; Eleven Bio) is administered intravenously. In some embodiments, EB-007 (EBI-029; Eleven Bio) is administered subcutaneously.

In some embodiments, the anti-IL-6 antibody is FM101 (Femta Pharmaceuticals, Lonza). In some embodiments, FM101 (Femta Pharmaceuticals, Lonza) is administered intravenously. In some embodiments, FM101 (Femta Pharmaceuticals, Lonza) is administered subcutaneously.

In some embodiments, the IL-6 antagonist is an anti-IL-6R antibody.

In some embodiments, the anti-IL-6R antibody is tocilizumab. In some embodiments, tocilizumab is administered intravenously at a monthly equivalent dose of 50-500 mg. In some embodiments, tocilizumab is administered intravenously at a monthly equivalent dose of about 50 mg. In some embodiments, tocilizumab is administered intravenously at a monthly equivalent dose of about 100 mg. In some embodiments, tocilizumab is administered intravenously at a monthly equivalent dose of about 150 mg. In some embodiments, tocilizumab is administered intravenously at a monthly equivalent dose of about 250 mg. In some embodiments, tocilizumab is administered intravenously at a monthly equivalent dose of about 350 mg. In some embodiments, tocilizumab is administered intravenously at a monthly equivalent dose of about 500 mg. In some embodiments, tocilizumab is administered subcutaneously at a monthly equivalent dose of 80-800 mg. In some embodiments, tocilizumab is administered subcutaneously at a monthly equivalent dose of about 80 mg. In some embodiments, tocilizumab is administered subcutaneously at a monthly equivalent dose of about 160 mg. In some embodiments, tocilizumab is administered subcutaneously at a monthly equivalent dose of about 240 mg. In some embodiments, tocilizumab is administered subcutaneously at a monthly equivalent dose of about 400 mg. In some embodiments, tocilizumab is administered subcutaneously at a monthly equivalent dose of about 560 mg. In some embodiments, tocilizumab is administered subcutaneously at a monthly equivalent dose of about 800 mg.

In some embodiments, the anti-IL-6R antibody is sarilumab. In some embodiments, sarilumab is administered intravenously at a monthly equivalent dose of 12-120 mg. In some embodiments, sarilumab is administered intravenously at a monthly equivalent dose of about 12 mg. In some embodiments, sarilumab is administered intravenously at a monthly equivalent dose of about 24 mg. In some embodiments, sarilumab is administered intravenously at a monthly equivalent dose of about 48 mg. In some embodiments, sarilumab is administered intravenously at a monthly equivalent dose of about 60 mg. In some embodiments, sarilumab is administered intravenously at a monthly equivalent dose of about 72 mg. In some embodiments, sarilumab is administered intravenously at a monthly equivalent dose of about 120 mg. In some embodiments, sarilumab is administered subcutaneously at a monthly equivalent dose of 20-200 mg. In some embodiments, sarilumab is administered subcutaneously at a monthly equivalent dose of about 20 mg. In some embodiments, sarilumab is administered subcutaneously at a monthly equivalent dose of about 40 mg. In some embodiments, sarilumab is administered subcutaneously at a monthly equivalent dose of about 80 mg. In some embodiments, sarilumab is administered subcutaneously at a monthly equivalent dose of about 100 mg. In some embodiments, sarilumab is administered subcutaneously at a monthly equivalent dose of about 120 mg. In some embodiments, sarilumab is administered subcutaneously at a monthly equivalent dose of about 200 mg.

In some embodiments, the anti-IL-6R antibody is vobarilizumab. In some embodiments, vobarilizumab is administered intravenously at a monthly equivalent dose of 4-120 mg. In some embodiments, vobarilizumab is administered intravenously at a monthly equivalent dose of about 4 mg. In some embodiments, vobarilizumab is administered intravenously at a monthly equivalent dose of about 6 mg. In some embodiments, vobarilizumab is administered intravenously at a monthly equivalent dose of about 30 mg. In some embodiments, vobarilizumab is administered intravenously at a monthly equivalent dose of about 60 mg. In some embodiments, vobarilizumab is administered intravenously at a monthly equivalent dose of about 84 mg. In some embodiments, vobarilizumab is administered intravenously at a monthly equivalent dose of about 120 mg. In some embodiments, vobarilizumab is administered subcutaneously at a monthly equivalent dose of 7-200 mg. In some embodiments, vobarilizumab is administered subcutaneously at a monthly equivalent dose of about 7 mg. In some embodiments, vobarilizumab is administered subcutaneously at a monthly equivalent dose of about 10 mg. In some embodiments, vobarilizumab is administered subcutaneously at a monthly equivalent dose of about 50 mg. In some embodiments, vobarilizumab is administered subcutaneously at a monthly equivalent dose of about 100 mg. In some embodiments, vobarilizumab is administered subcutaneously at a monthly equivalent dose of about 140 mg. In some embodiments, vobarilizumab is administered subcutaneously at a monthly equivalent dose of about 200 mg.

In some embodiments, the IL-6 antagonist is a JAK inhibitor. In some embodiments, the IL-6 antagonist is a STAT3 inhibitor.

In some embodiments, the patient has a hepcidin-mediated disorder.

In some embodiments, the patient has kidney disease. In some embodiments, the patient has chronic kidney disease. In some embodiments, the patient has KDOQI stage 1-5 chronic kidney disease. In some embodiments, the patient has KDOQI stage 3-5 chronic kidney disease. In some embodiments, the patient is not on dialysis. In some embodiments, the patient has KDOQI stage 5 chronic kidney disease. In some embodiments, the patient is on dialysis. In some embodiments, the patient has cardiorenal syndrome (CRS). In some embodiments, the patient has CRS Type 4.

In some embodiments, the patient has cardiovascular disease. In some embodiments, the patient has diuretic resistant heart failure. In some embodiments, the patient has congestive heart failure (CHF). In some embodiments, the patient has congestive heart failure (CHF) with reduced ejection fraction. In some embodiments, the patient has congestive heart failure (CHF) with mid-range ejection fraction. In some embodiments, the patient has congestive heart failure (CHF) with preserved ejection fraction. In some embodiments, the patient has acute coronary syndrome. In some embodiments, the patient has atherosclerosis.

In some embodiments, the patient has anemia. In some embodiments, the patient has anemia of chronic disease. In some embodiments, the patient has iron-refractory iron-deficiency anemia (IRIDA).

In some embodiments, the patient has diabetes. In some embodiments, the patient has type II diabetes. In some embodiments, the patient has insulin-resistant diabetes.

In some embodiments, the patient has liver disease. In some embodiments, the patient has non-alcoholic steatohepatitis (NASH).

In some embodiments, the patient has osteoporosis.

In some embodiments, the patient has depression.

In some embodiments, the patient has asthma.

In some embodiments, the patient has neuroinflammatory disorder. In some embodiments, the patient has Alzheimer's disease. In some embodiments, the patient has Parkinson's disease. In some embodiments, the patient has multiple sclerosis. In some embodiments, the patient has amyotrophic lateral sclerosis (ALS).

In some embodiments, the patient has age-related macular degeneration (AMD).

In some embodiments, the patient has cancer. In some embodiments, the cancer is selected from the group consisting of: solid tumors, small cell lung cancer, non-small cell lung cancer, hematological cancer, multiple myeloma, leukemia, chronic lymphocytic leukemia (CLL), chronic myeloid leukemia (CML), lymphomas, Hodgkin's lymphoma and hepatic adenoma.

In some embodiments, patient has skin disease.

In some embodiments, the method prevents aging in the patient.

In another aspect, methods for treating inflammation in a patient with cardiovascular disease are provided herein. The methods comprise: administering an IL-6 antagonist to a patient with cardiovascular disease and CRP level greater than 2 mg/L at a dose that is sufficient to reduce CRP levels to 2 mg/L or less without causing neutropenia.

In some embodiments, the IL-6 antagonist is administered at a monthly equivalent dose that is no more than 30% of the monthly equivalent dose for treating rheumatoid arthritis with the same IL-6 antagonist. In some embodiments, the IL-6 antagonist is administered at a monthly equivalent dose that is no more than 20% of the monthly equivalent dose for treating rheumatoid arthritis with the same IL-6 antagonist. In some embodiments, the IL-6 antagonist is administered at a monthly equivalent dose that is no more than 10% of the monthly equivalent dose for treating rheumatoid arthritis with the same IL-6 antagonist.

In another aspect, methods for treating inflammation in a patient with chronic kidney disease (CKD) are provided herein. The methods comprise: administering an IL-6 antagonist to a patient with CKD and a CRP level greater than 2 mg/L at a dose that is sufficient to reduce CRP levels to 2 mg/L or less without causing neutropenia.

In some embodiments, the IL-6 antagonist is administered at a monthly equivalent dose that is no more than 30% of the monthly equivalent dose for treating rheumatoid arthritis with the same IL-6 antagonist. In some embodiments, the IL-6 antagonist is administered at a monthly equivalent dose that is no more than 20% of the monthly equivalent dose for treating rheumatoid arthritis with the same IL-6 antagonist. In some embodiments, the IL-6 antagonist is administered at a monthly equivalent dose that is no more than 10% of the monthly equivalent dose for treating rheumatoid arthritis with the same IL-6 antagonist.

### BRIEF DESCRIPTION OF THE DRAWINGS

These and other features, aspects, and advantages of the present invention will become better understood with regard to the following description, and accompanying drawings, where:

FIG. 1 presents the dose escalation schematic for the phase 1/phase 2 randomized, double-blind, placebo-controlled trial of COR-001 in hemodialysis patients described in Example 1.

FIG. 2 shows the timeline and the efficacy analysis of the treatment phase and the safety follow-up phase.

FIGS. 3A and 3B show the results of C-reactive protein (CRP) responder analysis after treatment with COR-001 (anti-IL-6) or canakinumab (anti-IL1β). FIG. 3A shows the C-reactive protein responder rate after intravenous treatment with COR-001 in patients with stage 5 chronic kidney disease who were on dialysis in the clinical trial described in Example 1. The baseline hsCRP was 12.4 mg/L. Responder was defined as Week 12 average hsCRP<2 mg/L. FIG. 3B shows the C-reactive protein responder rate after treatment with canakinumab in the CANTOS trial, as described in the research literature. The baseline hsCRP was 5.5 mg/L. Responder was defined as 3-month hsCRP<2 mg/L.

FIG. 4 shows the results of hemoglobin responder analysis after treatment with COR-001 at doses of 2 mg, 6 mg, and 20 mg. Hemoglobin responder was defined as increase by 1 g/dL or more after Day 29. Investigators were not permitted to change ESA dosing until after Day 29.

FIG. 5 shows the effect of COR-001 on the diastolic cardiac parameter, NT-proBNP.

FIGS. 6A and 6B show the adverse responder rate for neutrophils and platelets. FIG. 6A shows the neutrophils adverse responder rate. An Adverse Responder was defined as Week 12 average neutrophils <2×10⁶/mL. FIG. 6B shows the platelets adverse responder rate. Adverse responder was defined as Week 12 average platelets <100×10⁶/mL.

### DETAILED DESCRIPTION

### 1. Definitions

Unless defined otherwise, all technical and scientific terms used herein have the meaning commonly understood by one of ordinary skill in the art to which the invention pertains.

As used herein, “interleukin 6 (IL-6)” or “IL-6 polypeptide” refers to a polypeptide or fragment thereof having at least about 85% or greater amino acid identity to the amino acid sequence provided at NCBI Accession No. NP_000591 and having IL-6 biological activity. IL-6 is a pleotropic cytokine with multiple biologic functions. Exemplary IL-6 biological activities include immunostimulatory and pro-inflammatory activities. An exemplary IL-6 amino acid sequence is provided below:

(SEQ ID NO: 1)

| | |
|---|---|
| 1 | MCVGARRLGR GPCAALLLLG LGLSTVTGLH CVGDTYPSND |
| | RCCHECRPGN GMVSRCSRSQ |
| 61 | NTVCRPCGPG FYNDVVSSKP CKPCTWCNLR SGSERKQLCT |
| | ATQDTVCRCR AGTQPLDSYK |
| 121 | PGVDCAPCPP GHFSPGDNQA CKPWTNCTLA GKHTLQPASN |
| | SSDAICEDRD PPATQPQETQ |
| 181 | GPPARPITVQ PTEAWPRTSQ GPSTRPVEVP GGRAVAAILG |
| | LGLVLGLLGP LAILLALYLL |
| 241 | RRDQRLPPDA HKPPGGGSFR TPIQEEQADA HSTLAKI |

As used herein, “interleukin 6 (IL-6) nucleic acid” refers to a polynucleotide encoding an interleukin 6 (IL-6) polypeptide. An exemplary interleukin 6 (IL-6) nucleic acid sequence is provided at NCBI Accession No. NM_000600. The exemplary sequence at NCBI Accession No. NM_000600 is provided below:

(SEQ ID NO: 2)

| | |
|---|---|
| 1 | AATATTAGAG TCTCAACCCC CAATAAATAT AGGACTGGAG |
| | ATGTCTGAGG CTCATTCTGC |
| 61 | CCTCGAGCCC ACCGGGAACG AAAGAGAAGC TCTATCTCCC |
| | CTCCAGGAGC CCAGCTATGA |
| 121 | ACTCCTTCTC CACAAGCGCC TTCGGTCCAG TTGCCTTCTC |
| | CCTGGGGCTG CTCCTGGTGT |
| 181 | TGCCTGCTGC CTTCCCTGCC CCAGTACCCC CAGGAGAAGA |
| | TTCCAAAGAT GTAGCCGCCC |
| 241 | CACACAGACA GCCACTCACC TCTTCAGAAC GAATTGACAA |
| | ACAAATTCGG TACATCCTCG |
| 301 | ACGGCATCTC AGCCCTGAGA AAGGAGACAT GTAACAAGAG |
| | TAACATGTGT GAAAGCAGCA |
| 361 | AAGAGGCACT GGCAGAAAAC AACCTGAACC TTCCAAAGAT |
| | GGCTGAAAAA GATGGATGCT |
| 421 | TCCAATCTGG ATTCAATGAG GAGACTTGCC TGGTGAAAAT |
| | CATCACTGGT CTTTTGGAGT |
| 481 | TTGAGGTATA CCTAGAGTAC CTCCAGAACA GATTTGAGAG |
| | TAGTGAGGAA CAAGCCAGAG |
| 541 | CTGTGCAGAT GAGTACAAAA GTCCTGATCC AGTTCCTGCA |
| | GAAAAAGGCA AAGAATCTAG |
| 601 | ATGCAATAAC CACCCCTGAC CCAACCACAA ATGCCAGCCT |
| | GCTGACGAAG CTGCAGGCAC |
| 661 | AGAACCAGTG GCTGCAGGAC ATGACAACTC ATCTCATTCT |
| | GCGCAGCTTT AAGGAGTTCC |
| 721 | TGCAGTCCAG CCTGAGGGCT CTTCGGCAAA TGTAGCATGG |
| | GCACCTCAGA TTGTTGTTGT |
| 781 | TAATGGGCAT TCCTTCTTCT GGTCAGAAAC CTGTCCACTG |
| | GGCACAGAAC TTATGTTGTT |
| 841 | CTCTATGGAG AACTAAAAGT ATGAGCGTTA GGACACTATT |
| | TTAATTATTT TTAATTTATT |
| 901 | AATATTTAAA TATGTGAAGC TGAGTTAATT TATGTAAGTC |
| | ATATTTATAT TTTTAAGAAG |
| 961 | TACCACTTGA AACATTTTAT GTATTAGTTT TGAAATAATA |
| | ATGGAAAGTG GCTATGCAGT |
| 1021 | TTGAATATCC TTTGTTTCAG AGCCAGATCA TTTCTTGGAA |
| | AGTGTAGGCT TACCTCAAAT |
| 1081 | AAATGGCTAA CTTATACATA TTTTTAAAGA AATATTTATA |
| | TTGTATTTAT ATAATGTATA |
| 1141 | AATGGTTTTT ATACCAATAA ATGGCATTTT AAAAAATTCA |
| | GCAAAAAAAA AAAAAAAAAA |
| 1201 | A |

As used herein, “interleukin 6 receptor (IL-6R) complex” refers to a protein complex comprising an IL-6 receptor subunit alpha (IL-6Rα) and interleukin 6 signal transducer glycoprotein 130 (gp130), also termed interleukin 6 receptor subunit β (IL-6Rβ).

As used herein, “interleukin 6 receptor subunit α (IL-6Rα) polypeptide” refers to a polypeptide or fragment thereof having at least about 85% or greater amino acid identity to the amino acid sequence provided at NCBI Accession No. NP_000556 or NP_852004 and having IL-6 receptor biological activity. Exemplary IL-6Rα biological activities include binding to IL-6, binding to glycoprotein 130 (gp130), and regulation of cell growth and differentiation. An exemplary IL-6R sequence is provided below:

(SEQ ID NO: 3)

| | |
|---|---|
| 1 | MLAVGCALLA ALLAAPGAAL APRRCPAQEV ARGVLTSLPG |
| | DSVTLTCPGV EPEDNATVHW |
| 61 | VLRKPAAGSH PSRWAGMGRR LLLRSVQLHD SGNYSCYRAG |
| | RPAGTVHLLV DVPPEEPQLS |
| 121 | CFRKSPLSNV VCEWGPRSTP SLTTKAVLLV RKFQNSPAED |
| | FQEPCQYSQE SQKFSCQLAV |
| 181 | PEGDSSFYIV SMCVASSVGS KFSKTQTFQG CGILQPDPPA |
| | NITVTAVARN PRWLSVTWQD |
| 241 | PHSWNSSFYR LRFELRYRAE RSKTFTTWMV KDLQHHCVIH |
| | DAWSGLRHVV QLRAQEEFGQ |
| 301 | GEWSEWSPEA MGTPWTESRS PPAENEVSTP MQALTTNKDD |
| | DNILFRDSAN ATSLPVQDSS |
| 361 | SVPLPTFLVA GGSLAFGTLL CIAIVLRFKK TWKLRALKEG |
| | KTSMHPPYSL GQLVPERPRP |
| 421 | TPVLVPLISP PVSPSSLGSD NTSSHNRPDA RDPRSPYDIS |
| | NTDYFFPR |

As used herein, “glycoprotein 130 (gp130)” or “interleukin 6 receptor subunit β (IL-6Rβ) polypeptide” refers to a polypeptide or fragment thereof having at least about 85% or greater amino acid identity to the amino acid sequence provided at NCBI Accession No. NP_002175, NP_786943, or NP_001177910 and having IL-6 receptor biological activity. Exemplary IL-6Rβ biological activities include binding to IL-6Rα, IL-6 receptor signaling activity, and regulation of cell growth, differentiation, hepcidin expression etc. An exemplary IL-6Rβ sequence is provided below:

(SEQ ID NO: 4)

| | |
|---|---|
| 1 | MLTLQTWLVQ ALFIFLTTES TGELLDPCGY ISPESPVVQL |
| | HSNFTAVCVL KEKCMDYFHV |
| 61 | NANYIVWKTN HFTIPKEQYT IINRTASSVT FTDIASLNIQ |
| | LTCNILTFGQ LEQNVYGITI |
| 121 | ISGLPPEKPK NLSCIVNEGK KMRCEWDGGR ETHLETNFTL |
| | KSEWATHKFA DCKAKRDTPT |
| 181 | SCTVDYSTVY FVNIEVWVEA ENALGKVTSD HINFDPVYKV |
| | KPNPPHNLSV INSEELSSIL |
| 241 | KLTWTNPSIK SVIILKYNIQ YRTKDASTWS QIPPEDTAST |
| | RSSFTVQDLK PFTEYVFRIR |
| 301 | CMKEDGKGYW SDWSEEASGI TYEDRPSKAP SFWYKIDPSH |
| | TQGYRTVQLV WKTLPPFEAN |
| 361 | GKILDYEVTL TRWKSHLQNY TVNATKLTVN LTNDRYLATL |
| | TVRNLVGKSD AAVLTIPACD |
| 421 | FQATHPVMDL KAFPKDNMLW VEWTTPRESV KKYILEWCVL |
| | SDKAPCITDW QQEDGTVHRT |
| 481 | YLRGNLAESK CYLITVTPVY ADGPGSPESI KAYLKQAPPS |
| | KGPTVRTKKV GKNEAVLEWD |
| 541 | QLPVDVQNGF IRNYTIFYRT IIGNETAVNV DSSHTEYTLS |
| | SLTSDTLYMV RMAAYTDEGG |
| 601 | KDGPEFTFTT PKFAQGEIEA IVVPVCLAFL LTTLLGVLFC |
| | FNKRDLIKKH IWPNVPDPSK |
| 661 | SHIAQWSPHT PPRHNFNSKD QMYSDGNFTD VSVVEIEAND |
| | KKPFPEDLKS LDLFKKEKIN |
| 721 | TEGHSSGIGG SSCMSSSRPS ISSSDENESS QNTSSTVQYS |
| | TVVHSGYRHQ VPSVQVFSRS |
| 781 | ESTQPLLDSE ERPEDLQLVD HVDGGDGILP RQQYFKQNCS |
| | QHESSPDISH FERSKQVSSV |
| 841 | NEEDFVRLKQ QISDHISQSC GSGQMKMFQE VSAADAFGPG |
| | TEGQVERFET VGMEAATDEG |
| 901 | MPKSYLPQTV RQGGYMPQ |

Unless otherwise specified, “IL-6 antagonist” refers an agent that is capable of decreasing the biological activity of IL-6. IL-6 antagonists include agents that decrease the level of IL-6 polypeptide in serum, including agents that decrease the expression of an IL-6 polypeptide or nucleic acid; agents that decrease the ability of IL-6 to bind to the IL-6R; agents that decrease the expression of the IL-6R; and agents that decrease signal transduction by the IL-6R receptor when bound by IL-6. In preferred embodiments, the IL-6 antagonist decreases IL-6 biological activity by at least about 10%, 20%, 30%, 50%, 70%, 80%, 90%, 95%, or even 100%. As further described below, IL-6 antagonists include IL-6 binding polypeptides, such as anti-IL-6 antibodies and antigen binding fragments or derivatives thereof; IL-6R binding polypeptides, such as anti-IL-6R antibodies and antigen binding fragments or derivatives thereof; and synthetic chemical molecules, such as JAK1 and JAK3 inhibitors.

The term “IL-6 antibody” or “anti-IL-6 antibody” refers to an antibody that specifically binds IL-6. Anti-IL-6 antibodies include monoclonal and polyclonal antibodies that are specific for IL-6, and antigen-binding fragments or derivatives thereof. IL-6 antibodies are described in greater detail below.

As used herein, the term “IL-6 mediated inflammation” or “IL-6 mediated inflammatory disorder” refers to inflammation or inflammation related disorder in which IL-6 is known or suspected to contribute to the etiology or symptoms of the inflammation.

The term “C-reactive protein” or “CRP” refers to a polypeptide or fragment thereof having at least about 85% or greater amino acid identity to the amino acid sequence provided at NCBI Accession No. NP_000558 and having complement activating activity. CRP levels increase in response to inflammation, and can be measured with an hsCRP (high-sensitivity C-reactive protein) test. An exemplary CRP sequence is provided below:

(SEQ ID NO: 5)

| | |
|---|---|
| 1 | MEKLLCFLVL TSLSHAFGQT DMSRKAFVFP KESDTSYVSL |
| | KAPLTKPLKA FTVCLHFYTE |
| 61 | LSSTRGYSIF SYATKRQDNE ILIFWSKDIG YSFTVGGSEI |
| | LFEVPEVTVA PVHICTSWES |
| 121 | ASGIVEFWVD GKPRVRKSLK KGYTVGAEAS IILGQEQDSF |
| | GGNFEGSQSL VGDIGNVNMW |
| 181 | DFVLSPDEIN TIYLGGPFSP NVLNWRALKY EVQGEVFTKP |
| | QLWP |

As used herein, “hepcidin” refers to a polypeptide having at least about 85% or greater amino acid identity to the amino acid sequence provided at NCBI Accession No. NP_066998 (“hepcidin preprotein”), or biologically active fragment thereof. Exemplary hepcidin biological activities include binding and reducing the levels of the iron export channel ferroportin, inhibiting iron transport, inhibiting intestinal iron absorption, and inhibiting iron release from macrophages and the liver. An exemplary hepcidin preprotein amino acid sequence is provided below:

(SEQ ID NO: 6)

| | |
|---|---|
| 1 | MALSSQIWAA CLLLLLLLAS LTSGSVFPQQ TGQLAELQPQ |
| | DRAGARASWM PMFQRRRRRD |
| 61 | THFPICIFCC GCCHRSKCGM CCKT |

With reference to the sequence above, hepcidin exists in various forms, including as a preprohormone (amino acids 25-84), prohormone (amino acids 25-84), and mature forms termed hepcidin-25 (amino acids 60-84), hepcidin-22 (amino acids 63-84), and hepcidin-20 (amino acids 65-84).

A “hepcidin-mediated disorder” is any disorder in which hepcidin expression contributes to the etiology of the disorder or any of its symptoms.

The term “immune suppression” or “immunosuppression” refers to a reduction of the activation or efficacy of the immune system. Immune suppression can be measured by the number of white blood cells, such as neutrophils.

As used herein, “neutrophil” of “neutrocyte” refers to a type of white blood cell that is an essential part of the innate immune system. The absolute neutrophil count (ANC) can be used in diagnosis and prognosis. Low neutrophil counts are termed neutropenia.

The term “agent” refers to any compound or composition suitable to be administered in therapy, and explicitly includes chemical compounds; proteins, including antibodies or antigen-binding fragments thereof; peptides; and nucleic acid molecules.

The term “subject” refers to a human or non-human mammal, including, but not limited to, bovine, equine, canine, ovine, feline, and rodent, including murine and rattus, subjects. A “patient” is a human subject in need of treatment.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

As used herein, “pre-treatment” means prior to the first administration of an IL-6 antagonist according the methods described herein. Pre-treatment does not exclude, and often includes, the prior administration of treatments other than an IL-6 antagonist.

As used herein, “post-treatment” means after the administration of an IL-6 antagonist according the methods described herein. Post-treatment includes after any administration of an IL-6 antagonist at any dosage described herein. Post-treatment also includes after the treatment phase of an IL-6 antagonist.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

The term “biological sample” refers to any tissue, cell, fluid, or other material derived from an organism (e.g., human subject). In certain embodiments, the biological sample is serum or blood.

Unless otherwise specified, antibody constant region residue numbering is according to the EU index as in Kabat.

### 2. Methods of Treating IL-6 Mediated Inflammation

In a first aspect, methods of treating IL-6-mediated inflammation in a patient are presented. The methods comprise administering an IL-6 antagonist to a patient with IL-6-mediated inflammation at a dose that is sufficient to reduce inflammation without causing immune suppression.

### 2.1. Pre-Treatment Serum CRP and IL-6 Levels

In the methods described herein, the patient has an IL-6-mediated inflammation.

In typical embodiments, the patient has elevated pre-treatment levels of C-reactive protein (CRP).

In some embodiments, the patient has a pre-treatment CRP level at least 2 mg/L. In some embodiments, the patient has a pre-treatment CRP level at least 2 mg/L, 2.5 mg/L, 3 mg/L, 3.5 mg/L, 4 mg/L, 4.5 mg/L, or 5 mg/L. In some embodiments, the patient has pre-treatment CRP levels at least 7.5 mg/L, 10 mg/L, 12.5 mg/L, or 15 mg/L. In various embodiments, the patient has a pre-treatment CRP level at least 2 mg/L. In various embodiments, the patient has a pre-treatment CRP level at least 2.5 mg/L. In various embodiments, the patient has a pre-treatment CRP level at least 5 mg/L. In various embodiments, the patient has a pre-treatment CRP level at least 7.5 mg/L. In various embodiments, the patient has a pre-treatment CRP level at least 10 mg/L. In various embodiments, the patient has a pre-treatment CRP level at least 12.5 mg/L. In various embodiments, the patient has a pre-treatment CRP level at least 15 mg/L.

In some embodiments of the methods described herein, the patient has elevated pre-treatment serum levels of IL-6.

In some embodiments, the patient has a pre-treatment serum IL-6 level of at least 2 pg/ml. In various embodiments, the patient has a pre-treatment serum IL-6 level of at least 2 pg/ml, at least 3 pg/ml, at least 4 pg/ml, at least 5 pg/ml, at least 6 pg/ml, at least 7 pg/ml, at least 8 pg/ml, at least 9 pg/ml, at least 10 pg/ml, at least 11 pg/ml, at least 12 pg/ml, at least 13 pg/ml, at least 14 pg/ml, or at least 15 pg/ml. In certain embodiments, the patient has a pre-treatment serum IL-6 level of at least 2 pg/ml. In certain embodiments, the patient has a pre-treatment serum IL-6 level of at least 2.5 pg/ml. In certain embodiments, the patient has a pre-treatment serum IL-6 level of at least 4 pg/ml. In certain embodiments, the patient has a pre-treatment serum IL-6 level of at least 5 pg/ml. In certain embodiments, the patient has a pre-treatment serum IL-6 level of at least 7.5 pg/ml. In certain embodiments, the patient has a pre-treatment serum IL-6 level of at least 10 pg/ml. In certain embodiments, the patient has a pre-treatment serum IL-6 level of at least 12.5 pg/ml. In certain embodiments, the patient has a pre-treatment serum IL-6 level of at least 15 pg/ml.

In some embodiments, the patient has elevated pre-treatment serum levels of CRP and elevated pre-treatment IL-6 levels. In certain embodiments, the patient has a pre-treatment serum IL-6 level of at least 2 pg/ml and a pre-treatment CRP level at least 2 mg/L. In certain embodiments, the patient has a pre-treatment serum IL-6 level of at least 2 pg/ml and a pre-treatment CRP level at least 2.5 mg/L. In certain embodiments, the patient has a pre-treatment serum IL-6 level of at least 2 pg/ml and a pre-treatment CRP level at least 5 mg/L. In certain embodiments, the patient has a pre-treatment serum IL-6 level of at least 2 pg/ml and a pre-treatment CRP level at least 10 mg/L. In certain embodiments, the patient has a pre-treatment serum IL-6 level of at least 4 pg/ml and a pre-treatment CRP level at least 2 mg/L. In certain embodiments, the patient has a pre-treatment serum IL-6 level of at least 4 pg/ml and a pre-treatment CRP level at least 2.5 mg/L. In certain embodiments, the patient has a pre-treatment serum IL-6 level of at least 4 pg/ml and a pre-treatment CRP level at least 5 mg/L. In certain embodiments, the patient has a pre-treatment serum IL-6 level of at least 4 pg/ml and a pre-treatment CRP level at least 10 mg/L. In certain embodiments, the patient has a pre-treatment serum IL-6 level of at least 5 pg/ml and a pre-treatment CRP level at least 2 mg/L. In certain embodiments, the patient has a pre-treatment serum IL-6 level of at least 5 pg/ml and a pre-treatment CRP level at least 2.5 mg/L. In certain embodiments, the patient has a pre-treatment serum IL-6 level of at least 5 pg/ml and a pre-treatment CRP level at least 5 mg/L. In certain embodiments, the patient has a pre-treatment serum IL-6 level of at least 5 pg/ml and a pre-treatment CRP level at least 10 mg/L. In certain embodiments, the patient has a pre-treatment serum IL-6 level of at least 10 pg/ml and a pre-treatment CRP level at least 2 mg/L. In certain embodiments, the patient has a pre-treatment serum IL-6 level of at least 10 pg/ml and a pre-treatment CRP level at least 2.5 mg/L. In certain embodiments, the patient has a pre-treatment serum IL-6 level of at least 10 pg/ml and a pre-treatment CRP level at least 5 mg/L. In certain embodiments, the patient has a pre-treatment serum IL-6 level of at least 10 pg/ml and a pre-treatment CRP level at least 10 mg/L.

### 2.2. Reduction of IL-6 and C-Reactive Protein (CRP)

In typical embodiments, the IL-6 antagonist is administered at a dose sufficient to reduce the patient's free serum IL-6 levels below pre-treatment levels.

In some embodiments, the free serum IL-6 level is decreased by at least 10% as compared to pre-treatment levels. In various embodiments, the free serum IL-6 level is decreased by at least 20%, 30%, 40%, 50%, 60%, 70%, 80%, or 90% as compared to pre-treatment levels. In certain embodiments, the free serum IL-6 level is decreased by at least 20% as compared to pre-treatment levels. In certain embodiments, the free serum IL-6 level is decreased by at least 30% as compared to pre-treatment levels. In certain embodiments, the free serum IL-6 level is decreased by at least 40% as compared to pre-treatment levels. In certain embodiments, the free serum IL-6 level is decreased by at least 50% as compared to pre-treatment levels. In certain embodiments, the free serum IL-6 level is decreased by at least 60% as compared to pre-treatment levels. In certain embodiments, the free serum IL-6 level is decreased by at least 70% as compared to pre-treatment levels. In certain embodiments, the free serum IL-6 level is decreased by at least 80% as compared to pre-treatment levels. In certain embodiments, the free serum IL-6 level is decreased by at least 90% as compared to pre-treatment levels.

In some embodiments, the IL-6 antagonist is administered at a dose sufficient to reduce the patient's CRP levels below pre-treatment levels. In some embodiments, the IL-6 mediated inflammation is measured by the CRP levels.

In certain embodiments, the post-treatment CRP level is no more than 5 mg/L. In certain embodiments, the post-treatment CRP level is no more than 2.5 mg/L. In certain embodiments, the post-treatment CRP level is no more than 2 mg/L. In certain embodiments, the post-treatment CRP level is no more than 1 mg/L.

In some embodiments, the CRP level is decreased by at least 10% as compared to pre-treatment levels. In various embodiments, the CRP level is decreased by at least 20%, 30%, 40%, 50%, 60%, 70%, 80%, or 90% as compared to pre-treatment levels. In certain embodiments, the CRP level is decreased by at least 20% as compared to pre-treatment levels. In certain embodiments, the CRP level is decreased by at least 30% as compared to pre-treatment levels. In certain embodiments, the CRP level is decreased by at least 40% as compared to pre-treatment levels. In certain embodiments, the CRP level is decreased by at least 50% as compared to pre-treatment levels. In certain embodiments, the CRP level is decreased by at least 60% as compared to pre-treatment levels. In certain embodiments, the CRP level is decreased by at least 70% as compared to pre-treatment levels. In certain embodiments, the CRP level is decreased by at least 80% as compared to pre-treatment levels. In certain embodiments, the CRP level is decreased by at least 90% as compared to pre-treatment levels.

### 2.3. Neutrophil Level

### 2.3.1. Absolute Neutrophil Count (ANC)

In the methods described herein, the IL-6 antagonist is administered at a dose sufficient to reduce inflammation without causing immune suppression.

In some embodiments, the immune suppression of the patient is measured by Absolute Neutrophil Count (ANC).

In some embodiments, the post-treatment ANC is at least 300 cells/μL. In various embodiments, the post-treatment ANC is at least 500 cells/μL, 600 cells/μL, 700 cells/μL, 800 cells/μL, 900 cells/μL, 1000 cells/μL, 1100 cells/μL, 1200 cells/μL, 1300 cells/μL, 1400 cells/μL, 1500 cells/μL, 1600 cells/μL, 1700 cells/μL, 1800 cells/μL, 1900 cells/μL, or 2000 cells/μL. In certain embodiments, the post-treatment ANC is at least 500 cells/μL. In certain embodiments, the post-treatment ANC is at least 750 cells/μL. In certain embodiments, the post-treatment ANC is at least 1000 cells/μL. In certain embodiments, the post-treatment ANC is at least 1250 cells/μL. In certain embodiments, the post-treatment ANC is at least 1500 cells/μL. In certain embodiments, the post-treatment ANC is at least 1750 cells/μL. In certain embodiments, the post-treatment ANC is at least 2000 cells/μL.

In some embodiments, the ANC is decreased by no more than 2500 cells/μL as compared to pre-treatment levels. In various embodiments, the ANC is decreased by no more than 2000 cells/μL, 1900 cells/μL, 1800 cells/μL, 1700 cells/μL, 1600 cells/μL, 1500 cells/μL, 1400 cells/μL, 1300 cells/μL, 1200 cells/μL, 1100 cells/μL, 1000 cells/μL, 900 cells/μL, 800 cells/μL, 700 cells/μL, 600 cells/μL, or 500 cells/μL, as compared to pre-treatment levels. In certain embodiments, the ANC is decreased by no more than 2000 cells/μL as compared to pre-treatment levels. In certain embodiments, the ANC is decreased by no more than 1750 cells/μL as compared to pre-treatment levels. In certain embodiments, the ANC is decreased by no more than 1500 cells/μL as compared to pre-treatment levels. In certain embodiments, the ANC is decreased by no more than 1250 cells/μL as compared to pre-treatment levels. In certain embodiments, the ANC is decreased by no more than 1000 cells/μL as compared to pre-treatment levels. In certain embodiments, the ANC is decreased by no more than 750 cells/μL as compared to pre-treatment levels. In certain embodiments, the ANC is decreased by no more than 500 cells/μL as compared to pre-treatment levels.

In some embodiments, the ANC is decreased by no more than 70% as compared to pre-treatment levels. In various embodiments, the ANC is decreased by no more than 60%, 50%, 40%, 30%, 20%, 10%, or 5% as compared to pre-treatment levels. In certain embodiments, the ANC is decreased by no more than 60% as compared to pre-treatment levels. In certain embodiments, the ANC is decreased by no more than 50% as compared to pre-treatment levels. In certain embodiments, the ANC is decreased by no more than 40% as compared to pre-treatment levels. In certain embodiments, the ANC is decreased by no more than 30% as compared to pre-treatment levels. In certain embodiments, the ANC is decreased by no more than 20% as compared to pre-treatment levels. In certain embodiments, the ANC is decreased by no more than 10% as compared to pre-treatment levels. In certain embodiments, the ANC is decreased by no more than 5% as compared to pre-treatment levels.

In some embodiments, the ANC is not decreased as compared to pre-treatment levels.

### 2.4. Lipoprotein(a) Level

In some embodiments, the IL-6 antagonist is administered at a dose sufficient to reduce the patient's lipoprotein(a) levels below pre-treatment levels.

In some embodiments, the lipoprotein(a) level is decreased by at least 10% as compared to pre-treatment levels. In various embodiments, the lipoprotein(a) level is decreased by at least 20%, 30%, 40%, 50%, 60%, 70%, 80%, or 90% as compared to pre-treatment levels. In certain embodiments, the lipoprotein(a) level is decreased by at least 20% as compared to pre-treatment levels. In certain embodiments, the lipoprotein(a) level is decreased by at least 30% as compared to pre-treatment levels. In certain embodiments, the lipoprotein(a) level is decreased by at least 40% as compared to pre-treatment levels. In certain embodiments, the lipoprotein(a) level is decreased by at least 50% as compared to pre-treatment levels. In certain embodiments, the lipoprotein(a) level is decreased by at least 60% as compared to pre-treatment levels. In certain embodiments, the lipoprotein(a) level is decreased by at least 70% as compared to pre-treatment levels. In certain embodiments, the lipoprotein(a) level is decreased by at least 80% as compared to pre-treatment levels. In certain embodiments, the lipoprotein(a) level is decreased by at least 90% as compared to pre-treatment levels.

### 2.5. LDL Level

In some embodiments, the IL-6 antagonist is administered at a dose sufficient to reduce the patient's lipoprotein(a) levels without significantly increasing the patient's low-density lipoprotein (LDL) levels.

In some embodiments, the LDL level is increased by no more than 15% as compared to pre-treatment levels. In various embodiments, the LDL level is increased by no more than 12%, 10%, 8%, 6%, 5%, 4%, 3%, 2% or 1% as compared to pre-treatment levels. In certain embodiments, the LDL level is increased by no more than 12% as compared to pre-treatment levels. In certain embodiments, the LDL level is increased by no more than 10% as compared to pre-treatment levels. In certain embodiments, the LDL level is increased by no more than 8% as compared to pre-treatment levels. In certain embodiments, the LDL level is increased by no more than 6% as compared to pre-treatment levels. In certain embodiments, the LDL level is increased by no more than 5% as compared to pre-treatment levels. In certain embodiments, the LDL level is increased by no more than 4% as compared to pre-treatment levels. In certain embodiments, the LDL level is increased by no more than 3% as compared to pre-treatment levels. In certain embodiments, the LDL level is increased by no more than 2% as compared to pre-treatment levels. In certain embodiments, the LDL level is increased by no more than 1% as compared to pre-treatment levels.

In certain embodiments, the LDL level is not increased as compared to pre-treatment levels.

### 2.6. IL-6 Mediated Inflammatory Disorders

In the methods described herein, the patient has an IL-6 mediated inflammatory disorder.

### 2.6.1. Non-Hepcidin-Mediated Inflammatory Disorders

In various embodiments, the IL-6 mediated inflammatory disorder is not a hepcidin-mediated disorder. Hepcidin-mediated disorders are described in US 2017/0029499, the disclosure of which is incorporated herein by reference in its entirety.

### 2.6.2. Hepcidin-Mediated Inflammatory Disorders

In various embodiments, the IL-6 mediated inflammatory disorder is a hepcidin-mediated disorder. Hepcidin-mediated disorders are described in US 2017/0029499, the disclosure of which is incorporated herein by reference in its entirety. In particular embodiments, the patient has a hepcidin-mediated disorder and at least one copy of the major allele at the TMPRSS6 rs855791 SNP (amino acid 736A). In other embodiments, the patient has a hepcidin-mediated disorder and is homozygous for the minor allele at the TMPRSS6 rs855791 SNP (amino acid 736V). In certain embodiments, the patient has a hepcidin-mediated disorder and unknown genotype at the TMPRSS6 rs855791 SNP.

### 2.6.3. Non-Autoimmune Inflammatory Disorder

In various embodiments, the IL-6 mediated inflammatory disorder is a non-autoimmune IL-6 mediated inflammatory disorder. In particular embodiments, the patient has an IL-6 mediated disorder other than rheumatoid arthritis, giant cell arteritis, polyarticular juvenile idiopathic arthritis, or systemic juvenile idiopathic arthritis.

### 2.6.4. Kidney Disease

In various embodiments, the patient has kidney disease. In some embodiments, the kidney disease is chronic kidney disease (CKD).

In some embodiments, the patient has KDOQI stage 1-5 chronic kidney disease. In some embodiments, the patient has KDOQI stage 3-5 chronic kidney disease. In some embodiments, the patient has KDOQI stage 1 chronic kidney disease, KDOQI stage 2 chronic kidney disease, KDOQI stage 3 chronic kidney disease, KDOQI stage 4 chronic kidney disease, or KDOQI stage 5 chronic kidney disease. In certain embodiments, the patient has KDOQI stage 5 chronic kidney disease.

In some embodiments, the patient is on dialysis. In some embodiments, the patient is not on dialysis. In certain embodiment, the patient has KDOQI stage 3-5 chronic kidney disease, wherein the patient is not on dialysis. In certain embodiment, the patient has KDOQI stage 5 chronic kidney disease, wherein the patient is on dialysis.

In some embodiments, the patient has cardiorenal syndrome (CRS). In certain embodiments, the patient has CRS Type 4.

In some embodiments, the patient has been treated with dialysis.

### 2.6.5. Cardiovascular Disease

In various embodiments, the patient has cardiovascular disease.

In some embodiments, the patient has had a previous myocardial infarction. In particular embodiments, the patient has had a previous myocardial infarction and has a CRP level of 2 mg/L or more.

In certain embodiments, the patient has suffered a myocardial infarction within the 60 days prior to first administration of an IL-6 antagonist. In particular embodiments, the patient has suffered a myocardial infarction within the 30 days, 14 days, 7 days, 48 hours, or 24 hours prior to first administration of an IL-6 antagonist.

In some embodiments, the patient has atherosclerosis but has not had a myocardial infarction. In particular embodiments, the patient has atherosclerosis, has not had a myocardial infarction, and has a CRP level of 2 mg/L or more.

In some embodiments, the cardiovascular disease is congestive heart failure (CHF). In certain embodiments, the patient has congestive heart failure (CHF) with reduced ejection fraction. In certain embodiments, the patient has congestive heart failure (CHF) with mid-range ejection fraction. In certain embodiments, the patient has congestive heart failure (CHF) with preserved ejection fraction.

In various embodiments, the IL-6 mediated inflammatory disorder is heart failure that is not diuretic resistant. Diuretic resistant heart failure is described in WO 2018/144773, the disclosure of which is incorporated herein by reference in its entirety.

In some embodiments, the cardiovascular disease is diuretic resistant heart failure. Diuretic resistant heart failure is described in WO 2018/144773, the disclosure of which is incorporated herein by reference in its entirety.

In some embodiments, the cardiovascular disease is acute coronary syndrome.

In certain embodiments, the IL-6 antagonist is administered at a dose sufficient to reduce nonfatal myocardial infarction, nonfatal stroke, and/or cardiovascular death. In some embodiments, the IL-6 antagonist is administered at a dose sufficient to reduce the risk of heart failure. In some embodiments, the IL-6 antagonist is administered at a dose sufficient to increase cardiac function. In some embodiments, the IL-6 antagonist is administered at a dose sufficient to reduce fibrosis after acute myocardial infarction.

### 2.6.6. Anemia

In various embodiments, the patient has anemia.

In some embodiments, the patient has anemia of chronic disease. In some embodiments, the patient has iron-refractory iron-deficiency anemia (IRIDA).

In some of these embodiments, the patient has been treated with an erythropoiesis-stimulating agent (ESA). In some embodiments, the patient has been treated with iron supplementation. In some embodiments, the patient has been treated with transfusion of blood or packed red blood cells.

In some embodiments, the IL-6 antagonist is administered at a dose sufficient to reverse functional iron deficiency.

### 2.6.7. Diabetes

In some embodiments, the patient has diabetes. In certain embodiments, the patient has type II diabetes. In certain embodiments, the patient has insulin-resistant diabetes.

### 2.6.8. Liver Disease

In some embodiments, the patient has liver disease. In certain embodiments, the patient has non-alcoholic steatohepatitis (NASH).

### 2.6.9. Osteoporosis

In some embodiments, the patient has osteoporosis.

### 2.6.10. Depression

In some embodiments, the patient has depression.

### 2.6.11. Asthma

In some embodiments, the patient has asthma.

### 2.6.12. Neuroinflammatory Disorder

In some embodiments, the patient has neuroinflammatory disorder. In certain embodiments, the patient has Alzheimer's disease. In certain embodiments, the patient has Parkinson's disease. In certain embodiments, the patient has multiple sclerosis. In certain embodiments, the patient has amyotrophic lateral sclerosis (ALS).

### 2.6.13. Age-Related Macular Degeneration

In some embodiments, the patient has age-related macular degeneration (AMD).

### 2.6.14. Cancer

In various embodiments, the patient has cancer.

In some embodiments, the cancer is selected from the group consisting of: solid tumors, small cell lung cancer, non-small cell lung cancer, hematological cancer, multiple myeloma, leukemia, chronic lymphocytic leukemia (CLL), chronic myeloid leukemia (CML), lymphomas, and Hodgkin's lymphoma.

### 2.6.15. Skin Disease

In various embodiments, the patient has skin disease, such as atopic dermatitis or psoriasis.

### 2.6.16. Aging

In some embodiments, the method prevents aging in the patient.

### 2.7. IL-6 Antagonists

The IL-6 antagonist used in the methods described herein is capable of decreasing the biological activity of IL-6.

### 2.7.1. Anti-IL-6 Antibodies

In various embodiments, the IL-6 antagonist is an anti-IL-6 antibody or antigen-binding fragment or derivative thereof.

In typical embodiments, the anti-IL-6 antibody neutralizes the biological activity of IL-6. In some embodiments, the neutralizing antibody prevents binding of IL-6 to the IL-6 receptor.

In some embodiments, the IL-6 antagonist is an anti-IL-6 monoclonal antibody. In some embodiments, the IL-6 antagonist is a polyclonal composition comprising a plurality of species of anti-IL-6 antibodies, each of the plurality having unique CDRs.

In some embodiments, the anti-IL-6 antibody is a Fab, Fab′, F(ab′)₂, Fv, scFv, (scFv)₂, single chain antibody molecule, dual variable domain antibody, single variable domain antibody, linear antibody, or V domain antibody.

In some embodiments, the anti-IL-6 antibody comprises a scaffold. In certain embodiments, the scaffold is Fc, optionally human Fc. In some embodiments, the anti-IL-6 antibody comprises a heavy chain constant region of a class selected from IgG, IgA, IgD, IgE, and IgM. In certain embodiments, the anti-IL-6 antibody comprises a heavy chain constant region of the class IgG and a subclass selected from IgG1, IgG2, IgG3, and IgG4.

In some embodiments, the IL-6 antagonist is immunoconjugate or fusion protein comprising an IL-6 antigen-binding fragment.

In some embodiments, the antibody is bispecific or multispecific, with at least one of the antigen-binding portions having specificity for IL-6.

In some embodiments, the antibody is fully human. In some embodiments, the antibody is humanized. In some embodiments, the antibody is chimeric and has non-human V regions and human C region domains. In some embodiments, the antibody is murine.

In typical embodiments, the anti-IL-6 antibody has a K_{D} for binding human IL-6 of less than 100 nM. In some embodiments, the anti-IL-6 antibody has a K_{D} for binding human IL-6 of less than 75 nM, 50 nM, 25 nM, 20 nM, 15 nM, or 10 nM. In particular embodiments, the anti-IL-6 antibody has a K_{D} for binding human IL-6 of less than 5 nM, 4 nM, 3 nM, or 2 nM. In selected embodiments, the anti-IL-6 antibody has a K_{D} for binding human IL-6 of less than 1 nM, 750 pM, or 500 pM. In specific embodiments, the anti-IL-6 antibody has a K_{D} for binding human IL-6 of no more than 500 pM, 400 pM, 300 pM, 200 pM, or 100 pM.

In typical embodiments, the anti-IL-6 antibody has an elimination half-life following intravenous administration of at least 7 days. In certain embodiments, the anti-IL-6 antibody has an elimination half-life of at least 14 days, at least 21 days, or at least 30 days.

In some embodiments, the anti-IL-6 antibody has a human IgG constant region with at least one amino acid substitution that extends serum half-life as compared to the unsubstituted human IgG constant domain.

In certain embodiments, the IgG constant domain comprises substitutions at residues 252, 254, and 256, wherein the amino acid substitution at amino acid residue 252 is a substitution with tyrosine, the amino acid substitution at amino acid residue 254 is a substitution with threonine, and the amino acid substitution at amino acid residue 256 is a substitution with glutamic acid (“YTE”). See U.S. Pat. No. 7,083,784, incorporated herein by reference in its entirety. In certain extended half-life embodiments, the IgG constant domain comprises substitutions selected from T250Q/M428L (Hinton et al., J. Immunology 176:346-356 (2006)); N434A (Yeung et al., J. Immunology 182:7663-7671 (2009)); or T307A/E380A/N434A (Petkova et al., International Immunology, 18:1759-1769 (2006)).

In some embodiments, the elimination half-life of the anti-IL-6 antibody is increased by utilizing the FcRN-binding properties of human serum albumin. In certain embodiments, the antibody is conjugated to albumin (Smith et al., Bioconjug. Chem., 12:750-756 (2001)). In some embodiments, the anti-IL-6 antibody is fused to bacterial albumin-binding domains (Stork et al., Prot. Eng. Design Science 20:569-76 (2007)). In some embodiments, the anti-IL-6 antibody is fused to an albumin-binding peptide (Nguygen et al., Prot Eng Design Sel 19:291-297 (2006)). In some embodiments, the anti-IL-6 antibody is bispecific, with one specificity being to IL-6, and one specificity being to human serum albumin (Ablynx, WO 2006/122825 (bispecific Nanobody)).

In some embodiments, the elimination half-life of the anti-IL-6 antibody is increased by PEGylation (Melmed et al., Nature Reviews Drug Discovery 7:641-642 (2008)); by HPMA copolymer conjugation (Lu et al., Nature Biotechnology 17:1101-1104 (1999)); by dextran conjugation (Nuclear Medicine Communications, 16:362-369 (1995)); by conjugation with homo-amino-acid polymers (HAPs; HAPylation) (Schlapschy et al., Prot Eng Design Sel 20:273-284 (2007)); or by polysialylation (Constantinou et al., Bioconjug. Chem. 20:924-931 (2009)).

### 2.7.1.1. COR-001 and Derivatives

In certain preferred embodiments, the anti-IL-6 antibody or antigen-binding portion thereof comprises all six CDRs of COR-001. The COR-001 antibody (also known as MEDI5117) is described in WO 2010/088444 and US 2012/0034212, the disclosures of which are incorporated herein by reference in their entireties. In particular embodiments, the antibody or antigen-binding portion thereof comprises the COR-001 heavy chain V region and light chain V region. In specific embodiments, the antibody is the full-length COR-001 antibody. The COR-001 antibody has the following CDR and heavy and light chain sequences:

COR-001 VH CDR1
(SEQ ID NO: 7)
SNYMI
COR-001 VH CDR2
(SEQ ID NO: 8)
DLYYYAGDTYYADSVKG
COR-001 VH CDR3
(SEQ ID NO: 9)
WADDHPPWIDL
COR-001 VL CDR1
(SEQ ID NO: 10)
RASQGISSWLA
COR-001 VL CDR2
(SEQ ID NO: 11)
KASTLES
COR-001 VL CDR3
(SEQ ID NO: 12)
QQSWLGGS
COR-001 Heavy chain
(SEQ ID NO: 13)
EVQLVESGGGLVQPGGSLRLSCAASGFTISSNYMIWVRQAPGKGLEWVSD
LYYYAGDTYYADSVKGRFTMSRDISKNTVYLQMNSLRAEDTAVYYCARWA
DDHPPWIDLWGRGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLYITREPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
COR-001 Light chain
(SEQ ID NO: 14)
DIQMTQSPSTLSASVGDRVTITCRASQGISSWLAWYQQKPGKAPKVLIYK
ASTLESGVPSRFSGSGSGTEFTLTISSLQPDDFATYYCQQSWLGGSFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In various embodiments, the anti-IL-6 antibody is a derivative of COR-001.

In some embodiments, the COR-001 derivative includes one or more amino acid substitutions in the COR-001 heavy and/or light chain V regions.

In certain embodiments, the COR-001 derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the COR-001 anti-IL-6 antibody, while retaining specificity for human IL-6.

In certain embodiments, the COR-001 derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of COR-001. The percent sequence identity is determined using BLAST algorithms using default parameters.

In certain embodiments, the COR-001 derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of COR-001. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6).

### 2.7.1.2. Siltuximab and Derivatives

In certain embodiments, the anti-IL-6 antibody or antigen-binding portion thereof comprises all six CDRs of siltuximab. In particular embodiments, the antibody or antigen-binding portion thereof comprises the siltuximab heavy chain V region and light chain V region. In specific embodiments, the antibody is the full-length siltuximab antibody.

In various embodiments, the anti-IL-6 antibody is a derivative of siltuximab.

In some embodiments, the siltuximab derivative includes one or more amino acid substitutions in the siltuximab heavy and/or light chain V regions.

In certain embodiments, the siltuximab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the siltuximab anti-IL-6 antibody, while retaining specificity for human IL-6.

In certain embodiments, the siltuximab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of siltuximab. The percent sequence identity is determined using BLAST algorithms using default parameters.

In certain embodiments, the siltuximab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of siltuximab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6).

### 2.7.1.3. Gerilimzumab and Derivatives

In certain embodiments, the anti-IL-6 antibody or antigen-binding portion thereof comprises all six CDRs of gerilimzumab. In particular embodiments, the antibody or antigen-binding portion thereof comprises the gerilimzumab heavy chain V region and light chain V region. In specific embodiments, the antibody is the full-length gerilimzumab antibody.

In various embodiments, the anti-IL-6 antibody is a derivative of gerilimzumab.

In some embodiments, the gerilimzumab derivative includes one or more amino acid substitutions in the gerilimzumab heavy and/or light chain V regions.

In certain embodiments, the gerilimzumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the gerilimzumab anti-IL-6 antibody, while retaining specificity for human IL-6.

In certain embodiments, the gerilimzumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of gerilimzumab. The percent sequence identity is determined using BLAST algorithms using default parameters.

In certain embodiments, the gerilimzumab derivative comprises an amino acid sequence in which the CDRs comprise an amino acid sequence that is at least 45%, at least 50%, least 90%, at least 95%, or at least 99% identical to the amino acid sequence of the respective CDRs of gerilimzumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6).

### 2.7.1.4. Sirukumab and Derivatives

In certain embodiments, the anti-IL-6 antibody or antigen-binding portion thereof comprises all six CDRs of sirukumab. In particular embodiments, the antibody or antigen-binding portion thereof comprises the sirukumab heavy chain V region and light chain V region. In specific embodiments, the antibody is the full-length sirukumab antibody.

In various embodiments, the anti-IL-6 antibody is a derivative of sirukumab.

In some embodiments, the sirukumab derivative includes one or more amino acid substitutions in the sirukumab heavy and/or light chain V regions.

In certain embodiments, the sirukumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the sirukumab anti-IL-6 antibody, while retaining specificity for human IL-6.

In certain embodiments, the sirukumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of sirukumab. The percent sequence identity is determined using BLAST algorithms using default parameters.

In certain embodiments, the sirukumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of sirukumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6).

### 2.7.1.5. Clazakizumab and Derivatives

In certain embodiments, the anti-IL-6 antibody or antigen-binding portion thereof comprises all six CDRs of clazakizumab. In particular embodiments, the antibody or antigen-binding portion thereof comprises the clazakizumab heavy chain V region and light chain V region. In specific embodiments, the antibody is the full-length clazakizumab antibody.

In various embodiments, the anti-IL-6 antibody is a derivative of clazakizumab.

In some embodiments, the clazakizumab derivative includes one or more amino acid substitutions in the clazakizumab heavy and/or light chain V regions.

In certain embodiments, the clazakizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the clazakizumab anti-IL-6 antibody, while retaining specificity for human IL-6.

In certain embodiments, the clazakizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of clazakizumab. The percent sequence identity is determined using BLAST algorithms using default parameters.

In certain embodiments, the clazakizumab derivative comprises an amino acid sequence in which the CDRs comprise an amino acid sequence that is at least 45%, at least 50%, least 90%, at least 95%, or at least 99% identical to the amino acid sequence of the respective CDRs of clazakizumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6).

### 2.7.1.6. Olokizumab and Derivatives

In certain embodiments, the anti-IL-6 antibody or antigen-binding portion thereof comprises all six CDRs of olokizumab. In particular embodiments, the antibody or antigen-binding portion thereof comprises the olokizumab heavy chain V region and light chain V region. In specific embodiments, the antibody is the full-length olokizumab antibody.

In various embodiments, the anti-IL-6 antibody is a derivative of olokizumab.

In some embodiments, the olokizumab derivative includes one or more amino acid substitutions in the olokizumab heavy and/or light chain V regions.

In certain embodiments, the olokizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the olokizumab anti-IL-6 antibody, while retaining specificity for human IL-6.

In certain embodiments, the olokizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of olokizumab. The percent sequence identity is determined using BLAST algorithms using default parameters.

In certain embodiments, the olokizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of olokizumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6).

### 2.7.1.7. Other Anti-IL-6 Antibodies and Derivatives

In certain embodiments, the anti-IL-6 antibody or antigen-binding portion thereof comprises all six CDRs of an antibody selected from the group consisting of: VX30 (VOP-R003; Vaccinex), EB-007 (EBI-029; Eleven Bio), and FM101. In particular embodiments, the antibody or antigen-binding portion thereof comprises the heavy chain V region and light chain V region of an antibody selected from the group consisting of: VX30 (VOP-R003; Vaccinex), EB-007 (EBI-029; Eleven Bio), and FM101. In specific embodiments, the antibody is a full-length antibody selected from the group consisting of: VX30 (VOP-R003; Vaccinex), EB-007 (EBI-029; Eleven Bio), and FM101.

In various embodiments, the anti-IL-6 antibody is a derivative of an antibody selected from the group consisting of: VX30 (VOP-R003; Vaccinex), EB-007 (EBI-029; Eleven Bio), and FM101.

### 2.7.2. Anti-IL-6 Receptor Antibodies

In various embodiments, the IL-6 antagonist is an anti-IL-6 receptor (anti-IL-6R) antibody or antigen-binding fragment or derivative thereof.

In typical embodiments, the anti-IL-6R reduces the biological activity of IL-6 receptor.

In some embodiments, the IL-6 antagonist is an anti-IL-6R monoclonal antibody. In some embodiments, the IL-6 antagonist is a polyclonal composition comprising a plurality of species of anti-IL-6R antibodies, each of the plurality having unique CDRs.

In some embodiments, the anti-IL-6R antibody is a Fab, Fab′, F(ab′)₂, Fv, scFv, (scFv)₂, single chain antibody molecule, dual variable domain antibody, single variable domain antibody, linear antibody, or V domain antibody.

In some embodiments, the anti-IL-6R antibody comprises a scaffold. In certain embodiments, the scaffold is Fc, optionally human Fc. In some embodiments, the anti-IL-6R antibody comprises a heavy chain constant region of a class selected from IgG, IgA, IgD, IgE, and IgM. In certain embodiments, the anti-IL-6R antibody comprises a heavy chain constant region of the class IgG and a subclass selected from IgG1, IgG2, IgG3, and IgG4.

In some embodiments, the IL-6 antagonist is immunoconjugate or fusion protein comprising an IL-6R antigen-binding fragment.

In some embodiments, the antibody is bispecific or multispecific, with at least one of the antigen-binding portions having specificity for IL-6 receptor.

In some embodiments, the antibody is fully human. In some embodiments, the antibody is humanized. In some embodiments, the antibody is chimeric and has non-human V regions and human C region domains. In some embodiments, the antibody is murine.

In typical embodiments, the anti-IL-6R antibody has a K_{D} for binding human IL-6 receptor of less than 100 nM. In some embodiments, the anti-IL-6R antibody has a K_{D} for binding human IL-6 receptor of less than 75 nM, 50 nM, 25 nM, 20 nM, 15 nM, or 10 nM. In particular embodiments, the anti-IL-6R antibody has a K_{D} for binding human IL-6 receptor of less than 5 nM, 4 nM, 3 nM, or 2 nM. In selected embodiments, the anti-IL-6R antibody has a K_{D} for binding human IL-6 receptor of less than 1 nM, 750 pM, or 500 pM. In specific embodiments, the anti-IL-6R antibody has a K_{D} for binding human IL-6 receptor of no more than 500 pM, 400 pM, 300 pM, 200 pM, or 100 pM.

In typical embodiments, the anti-IL-6R antibody has an elimination half-life following intravenous administration of at least 7 days. In certain embodiments, the anti-IL-6R antibody has an elimination half-life of at least 14 days, at least 21 days, or at least 30 days.

In some embodiments, the anti-IL-6R antibody has a human IgG constant region with at least one amino acid substitution that extends serum half-life as compared to the unsubstituted human IgG constant domain.

In certain embodiments, the IgG constant domain comprises substitutions at residues 252, 254, and 256, wherein the amino acid substitution at amino acid residue 252 is a substitution with tyrosine, the amino acid substitution at amino acid residue 254 is a substitution with threonine, and the amino acid substitution at amino acid residue 256 is a substitution with glutamic acid (“YTE”). See U.S. Pat. No. 7,083,784, incorporated herein by reference in its entirety. In certain extended half-life embodiments, the IgG constant domain comprises substitutions selected from T250Q/M428L (Hinton et al., J. Immunology 176:346-356 (2006)); N434A (Yeung et al., J. Immunology 182:7663-7671 (2009)); or T307A/E380A/N434A (Petkova et al., International Immunology, 18:1759-1769 (2006)).

In some embodiments, the elimination half-life of the anti-IL-6R antibody is increased by utilizing the FcRN-binding properties of human serum albumin. In certain embodiments, the antibody is conjugated to albumin (Smith et al., Bioconjug. Chem., 12:750-756 (2001)). In some embodiments, the anti-IL-6R antibody is fused to bacterial albumin-binding domains (Stork et al., Prot. Eng. Design Science 20:569-76 (2007)). In some embodiments, the anti-IL-6R antibody is fused to an albumin-binding peptide (Nguygen et al., Prot Eng Design Sel 19:291-297 (2006)). In some embodiments, the anti-IL-6R antibody is bispecific, with one specificity being to IL-6 receptor, and one specificity being to human serum albumin (Ablynx, WO 2006/122825 (bispecific Nanobody)).

In some embodiments, the elimination half-life of the anti-IL-6R antibody is increased by PEGylation (Melmed et al., Nature Reviews Drug Discovery 7:641-642 (2008)); by HPMA copolymer conjugation (Lu et al., Nature Biotechnology 17:1101-1104 (1999)); by dextran conjugation (Nuclear Medicine Communications, 16:362-369 (1995)); by conjugation with homo-amino-acid polymers (HAPs; HAPylation) (Schlapschy et al., Prot Eng Design Sel 20:273-284 (2007)); or by polysialylation (Constantinou et al., Bioconjug. Chem. 20:924-931 (2009)).

### 2.7.2.1. Tocilizumab and Derivatives

In certain embodiments, the anti-IL-6R antibody or antigen-binding portion thereof comprises all six CDRs of tocilizumab. In particular embodiments, the antibody or antigen-binding portion thereof comprises the tocilizumab heavy chain V region and light chain V region. In specific embodiments, the antibody is the full-length tocilizumab antibody.

In various embodiments, the anti-IL-6R antibody is a derivative of tocilizumab.

In some embodiments, the tocilizumab derivative includes one or more amino acid substitutions in the tocilizumab heavy and/or light chain V regions.

In certain embodiments, the tocilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the tocilizumab anti-IL-6R antibody, while retaining specificity for human IL-6 receptor.

In certain embodiments, the tocilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of tocilizumab. The percent sequence identity is determined using BLAST algorithms using default parameters.

In certain embodiments, the tocilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of tocilizumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6 receptor).

### 2.7.2.2. Sarilumab and Derivatives

In certain embodiments, the anti-IL-6R antibody or antigen-binding portion thereof comprises all six CDRs of sarilumab. In particular embodiments, the antibody or antigen-binding portion thereof comprises the sarilumab heavy chain V region and light chain V region. In specific embodiments, the antibody is the full-length sarilumab antibody.

In various embodiments, the anti-IL-6R antibody is a derivative of sarilumab.

In some embodiments, the sarilumab derivative includes one or more amino acid substitutions in the sarilumab heavy and/or light chain V regions.

In certain embodiments, the sarilumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the sarilumab anti-IL-6R antibody, while retaining specificity for human IL-6 receptor.

In certain embodiments, the sarilumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of sarilumab. The percent sequence identity is determined using BLAST algorithms using default parameters.

In certain embodiments, the sarilumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of sarilumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6 receptor).

### 2.7.2.3. Vobarilizumab and Derivatives

In certain embodiments, the anti-IL-6R antibody or antigen-binding portion thereof comprises all six CDRs of vobarilizumab. In particular embodiments, the antibody or antigen-binding portion thereof comprises the vobarilizumab heavy chain V region and light chain V region. In specific embodiments, the antibody is the full-length vobarilizumab antibody.

In various embodiments, the anti-IL-6R antibody is a derivative of vobarilizumab.

In some embodiments, the vobarilizumab derivative includes one or more amino acid substitutions in the vobarilizumab heavy and/or light chain V regions.

In certain embodiments, the vobarilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original V_{H} and/or V_{L} of the vobarilizumab anti-IL-6R antibody, while retaining specificity for human IL-6 receptor.

In certain embodiments, the vobarilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the V_{H} and V_{L} domain of vobarilizumab. The percent sequence identity is determined using BLAST algorithms using default parameters.

In certain embodiments, the vobarilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of vobarilizumab. The percent sequence identity is determined using BLAST algorithms using default parameters.

In certain embodiments, the V_{H} and/or V_{L} CDR derivatives comprise conservative amino acid substitutions at one or more predicted nonessential amino acid residues (i.e., amino acid residues which are not critical for the antibody to specifically bind to human IL-6 receptor).

### 2.7.2.4. Other Anti-IL-6R Antibodies and Derivatives

comprises all six CDRs of an antibody selected from the group consisting of: SA237 (Roche), NI-1201 (NovImmune), and an antibody described in US 2012/0225060. In particular embodiments, the antibody or antigen-binding portion thereof comprises the heavy chain V region and light chain V region of an antibody selected from the group consisting of: SA237 (Roche), NI-1201 (NovImmune), and an antibody described in US 2012/0225060. In specific embodiments, the antibody is a full-length selected from the group consisting of: SA237 (Roche), NI-1201 (NovImmune), and an antibody described in US 2012/0225060.

In various embodiments, the anti-IL-6R antibody is a derivative of an antibody selected from the group consisting of: SA237 (Roche), NI-1201 (NovImmune), or an antibody described in US 2012/0225060.

### 2.7.3. Anti-IL-6:IL-6R Complex Antibodies

In various embodiments, the IL-6 antagonist is an antibody specific for the complex of IL-6 and IL-6R. In certain embodiments, the antibody has the six CDRs of an antibody selected from those described in US 2011/0002936, which is incorporated herein by reference in its entirety.

### 2.7.4. JAK and STAT Inhibitors

In various embodiments, the IL-6 antagonist is an inhibitor of the JAK signaling pathway. In some embodiments, the JAK inhibitor is a JAK1-specific inhibitor. In some embodiments, the JAK inhibitor is a JAK3-specific inhibitor. In some embodiments, the JAK inhibitor is a pan-JAK inhibitor.

In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, INCB-039110, ABT-494, INCB-047986 and AC-410.

In various embodiments, the IL-6 antagonist is a STAT3 inhibitor. In a specific embodiment, the inhibitor is AZD9150 (AstraZeneca, Isis Pharmaceuticals), a STAT3 antisense molecule.

### 2.7.5. Additional IL-6 Antagonists

In various embodiments, the IL-6 antagonist is an antagonist peptide.

In certain embodiments, the IL-6 antagonist is C326 (an IL-6 inhibitor by Avidia, also known as AMG220), or FE301, a recombinant protein inhibitor of IL-6 (Ferring International Center S.A., Conaris Research Institute AG). In some embodiments, the anti-IL-6 antagonist comprises soluble gp130, FE301 (Conaris/Ferring).

### 2.8. Pharmaceutical Composition

The IL-6 antagonists used in the methods described herein can be formulated in any appropriate pharmaceutical composition for administration by any suitable route of administration. Suitable routes of administration include, but are not limited to, the intravitreal, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

### 2.9. Dosage Regimens

The IL-6 antagonist is administered at a dose sufficient to reduce inflammation without causing immune suppression.

### 2.9.1. Antibodies, Antigen-Binding Fragments, Peptides

In typical embodiments, antibody, antigen-binding fragments, and peptide IL-6 antagonists are administered parenterally.

In some parenteral embodiments, the IL-6 antagonist is administered intravenously. In certain intravenous embodiments, the IL-6 antagonist is administered as a bolus. In certain intravenous embodiments, the IL-6 antagonist is administered as an infusion. In certain intravenous embodiments, the IL-6 antagonist is administered as a bolus followed by infusion.

In some parenteral embodiments, the IL-6 antagonist is administered subcutaneously.

In various embodiments, the antibody, antigen-binding fragment, or peptide IL-6 antagonist is administered in a dose that is independent of patient weight or surface area (flat dose).

In some embodiments, the intravenous flat dose is 0.1 mg, 0.2 mg, 0.3 mg, 0.4 mg, 0.5 mg, 0.6 mg, 0.7 mg, 0.8 mg, 0.9 mg, or 1 mg. In some embodiments, the intravenous flat dose is 1 mg, 2 mg, 3 mg, 4 mg, 5 mg, 6 mg, 7 mg, 8 mg, 9 mg, or 10 mg. In some embodiments, the intravenous flat dose is 11 mg, 12 mg, 13 mg, 14 mg, 15 mg, 16 mg, 17 mg, 18 mg, 19 mg, or 20 mg. In some embodiments, the intravenous flat dose is 25 mg, 30 mg, 40 mg, or 50 mg. In some embodiments, the intravenous flat dose is 60 mg, 70 mg, 80 mg, 90 mg, or 100 mg. In some embodiments, the intravenous flat dose is 200 mg, 300 mg, 400 mg, or 500 mg. In some embodiments, the intravenous flat dose is 0.1-1 mg, 1-10 mg, 10-15 mg, 15-20 mg, 20-30 mg, 30-40 mg, or 40-50 mg. In some embodiments, the intravenous flat dose is 1-50 mg, 50-100 mg, or 100 mg-500 mg.

In some embodiments, the subcutaneous flat dose is 0.1 mg, 0.2 mg, 0.3 mg, 0.4 mg, 0.5 mg, 0.6 mg, 0.7 mg, 0.8 mg, 0.9 mg, or 1 mg. In some embodiments, the subcutaneous flat dose is 1 mg, 2 mg, 3 mg, 4 mg, 5 mg, 6 mg, 7 mg, 8 mg, 9 mg, or 10 mg. In some embodiments, the subcutaneous flat dose is 11 mg, 12 mg, 13 mg, 14 mg, 15 mg, 16 mg, 17 mg, 18 mg, 19 mg, or 20 mg. In some embodiments, the subcutaneous flat dose is 25 mg, 30 mg, 40 mg, or 50 mg. In some embodiments, the subcutaneous flat dose is 60 mg, 70 mg, 80 mg, 90 mg, or 100 mg. In some embodiments, the subcutaneous flat dose is 200 mg, 300 mg, 400 mg, or 500 mg. In some embodiments, the subcutaneous flat dose is 0.1-1 mg, 1-10 mg, 10-15 mg, 15-20 mg, 20-30 mg, 30-40 mg, or 40-50 mg. In some embodiments, the subcutaneous flat dose is 1-50 mg, 50-100 mg, or 100 mg-500 mg.

In various embodiments, the antibody, antigen-binding fragment, or peptide IL-6 antagonist is administered as a patient weight-based dose.

In some embodiments, the antagonist is administered at an intravenous dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg or 0.1 mg/kg. In some embodiments, the antagonist is administered at an intravenous dose of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg or 1.0 mg/kg. In some embodiments, the antagonist is administered at an intravenous dose of 1.5 mg/kg, 2 mg/kg, 2.5 mg/kg, 3 mg/kg, 3.5 mg/kg, 4 mg/kg, 4.5 mg/kg, or 5 mg/kg.

In some embodiments, the antagonist is administered at a subcutaneous dose of 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg or 0.1 mg/kg. In some embodiments, the antagonist is administered at a subcutaneous dose of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg or 1.0 mg/kg. In some embodiments, the antagonist is administered at a subcutaneous dose of 1.5 mg/kg, 2 mg/kg, 2.5 mg/kg, 3 mg/kg, 3.5 mg/kg, 4 mg/kg, 4.5 mg/kg, or 5 mg/kg.

In various intravenous embodiments, the IL-6 antagonist is administered once every 7 days, once every 14 days, once every 21 days, once every 28 days, or once a month. In various subcutaneous embodiments, the IL-6 antagonist is administered once every 14 days, once every 28 days, once a month, once every two months (every other month), or once every three months.

### 2.9.2. Small Molecule Inhibitors

In typical embodiments, small molecule JAK inhibitors and STAT inhibitors are administered orally.

In various embodiments, the inhibitor is administered once or twice a day at an oral dose of 0.1-1 mg, 1-10 mg, 10-20 mg, 20-30 mg, 30-40 mg, or 40-50 mg. In some embodiments, the inhibitor is administered once or twice a day at a dose of 50-60 mg, 60-70 mg, 70-80 mg, 80-90 mg, or 90-100 mg. In some embodiments, the inhibitor is administered at a dose of 0.1, 0.5, 1, 2, 5, 10, 15, 20, 25, 30, 35, 40, 45, or 50 mg PO once or twice a day. In some embodiments, the inhibitor is administered at a dose of 75 mg or 100 mg PO once or twice a day.

### 2.9.3. Monthly Equivalent Dose

In typical embodiments, the IL-6 antagonist is administered at a monthly equivalent dose that is less than the monthly equivalent dose for treating rheumatoid arthritis with the same IL-6 antagonist. “Monthly equivalent dose” is the calculated total dose administered per month, regardless of dose amount and dosage schedule.

In some embodiments, the IL-6 antagonist is administered at a monthly equivalent dose no more than 50% of a monthly equivalent dose for treating rheumatoid arthritis with the same IL-6 antagonist. In various embodiments, the IL-6 antagonist is administered at a monthly equivalent dose no more than 45%, 40%, 35%, 30%, 25%, 20%, 15%, 10%, or 5% of a monthly equivalent dose for treating rheumatoid arthritis with the same IL-6 antagonist. In certain embodiments, the IL-6 antagonist is administered at a monthly equivalent dose no more than 45% of a monthly equivalent dose for treating rheumatoid arthritis. In certain embodiments, the IL-6 antagonist is administered at a monthly equivalent dose no more than 40% of a monthly equivalent dose for treating rheumatoid arthritis. In certain embodiments, the IL-6 antagonist is administered at a monthly equivalent dose no more than 30% of a monthly equivalent dose for treating rheumatoid arthritis. In certain embodiments, the IL-6 antagonist is administered at a monthly equivalent dose no more than 25% of a monthly equivalent dose for treating rheumatoid arthritis. In certain embodiments, the IL-6 antagonist is administered at a monthly equivalent dose no more than 20% of a monthly equivalent dose for treating rheumatoid arthritis. In certain embodiments, the IL-6 antagonist is administered at a monthly equivalent dose no more than 15% of a monthly equivalent dose for treating rheumatoid arthritis. In certain embodiments, the IL-6 antagonist is administered at a monthly equivalent dose no more than 10% of a monthly equivalent dose for treating rheumatoid arthritis. In certain embodiments, the IL-6 antagonist is administered at a monthly equivalent dose no more than 5% of a monthly equivalent dose for treating rheumatoid arthritis.

In various embodiments, the IL-6 antagonist is administered at a monthly equivalent dose about 50%, 45%, 40%, 35%, 30%, 25%, 20%, 15%, 10%, or 5% of a monthly equivalent dose for treating rheumatoid arthritis with the same IL-6 antagonist. In certain embodiments, the IL-6 antagonist is administered at a monthly equivalent dose about 50% of a monthly equivalent dose for treating rheumatoid arthritis. In certain embodiments, the IL-6 antagonist is administered at a monthly equivalent dose about 40% of a monthly equivalent dose for treating rheumatoid arthritis. In certain embodiments, the IL-6 antagonist is administered at a monthly equivalent dose about 30% of a monthly equivalent dose for treating rheumatoid arthritis. In certain embodiments, the IL-6 antagonist is administered at a monthly equivalent dose about 25% of a monthly equivalent dose for treating rheumatoid arthritis. In certain embodiments, the IL-6 antagonist is administered at a monthly equivalent dose about 20% of a monthly equivalent dose for treating rheumatoid arthritis. In certain embodiments, the IL-6 antagonist is administered at a monthly equivalent dose about 15% of a monthly equivalent dose for treating rheumatoid arthritis. In certain embodiments, the IL-6 antagonist is administered at a monthly equivalent dose about 10% of a monthly equivalent dose for treating rheumatoid arthritis. In certain embodiments, the IL-6 antagonist is administered at a monthly equivalent dose about 5% of a monthly equivalent dose for treating rheumatoid arthritis.

In some embodiments, the IL-6 antagonist is the COR-001 antibody. In various embodiments, COR-001 is administered intravenously at a monthly equivalent dose of 0.5-50 mg, such as 0.5-1 mg, 0.5-2 mg, 0.5-5 mg, 0.5-10 mg, 0.5-20 mg, 0.5-30 mg, 0.5-40 mg, 1-2 mg, 1-5 mg, 1-10 mg, 1-20 mg, 1-30 mg, 1-40 mg, 1-50 mg, 2-5 mg, 2-10 mg, 2-20 mg, 2-30 mg, 2-40 mg, 2-50 mg, 5-10 mg, 5-20 mg, 5-30 mg, 5-40 mg, 5-50 mg, 10-20 mg, 10-30 mg, 10-40 mg, 10-50 mg, 20-30 mg, 20-40 mg, 20-50 mg, 30-40 mg, 30-50 mg, or 40-50 mg. In certain preferred embodiments, COR-001 is administered intravenously at a monthly equivalent dose of 2-40 mg.

In various embodiments, COR-001 is administered intravenously at a monthly equivalent dose of about 0.5 mg, 1 mg, 2 mg, 3 mg, 4 mg, 5 mg, 6 mg, 7 mg, 8 mg, 9 mg, 10 mg, 20 mg, 30 mg, 40 mg, or 50 mg. In certain embodiments, COR-001 is administered intravenously at a monthly equivalent dose of about 1 mg. In certain embodiments, COR-001 is administered intravenously at a monthly equivalent dose of about 2 mg. In certain embodiments, COR-001 is administered intravenously at a monthly equivalent dose of about 3 mg. In certain embodiments, COR-001 is administered intravenously at a monthly equivalent dose of about 4 mg. In certain embodiments, COR-001 is administered intravenously at a monthly equivalent dose of about 5 mg. In certain embodiments, COR-001 is administered intravenously at a monthly equivalent dose of about 6 mg. In certain embodiments, COR-001 is administered intravenously at a monthly equivalent dose of about 10 mg. In certain embodiments, COR-001 is administered intravenously at a monthly equivalent dose of about 12 mg. In certain embodiments, COR-001 is administered intravenously at a monthly equivalent dose of about 15 mg. In certain embodiments, COR-001 is administered intravenously at a monthly equivalent dose of about 20 mg. In certain embodiments, COR-001 is administered intravenously at a monthly equivalent dose of about 40 mg.

In various embodiments, COR-001 is administered subcutaneously at a monthly equivalent dose of 1-100 mg, such as 1-2 mg, 1-5 mg, 1-10 mg, 1-20 mg, 1-30 mg, 1-40 mg, 1-50 mg, 1-70 mg, 1-100 mg, 2-5 mg, 2-10 mg, 2-20 mg, 2-30 mg, 2-40 mg, 2-50 mg, 2-70 mg, 2-100 mg, 3-5 mg, 3-10 mg, 3-20 mg, 3-30 mg, 3-40 mg, 3-50 mg, 3-70 mg, 3-100 mg, 5-10 mg, 5-20 mg, 5-30 mg, 5-40 mg, 5-50 mg, 5-70 mg, 5-100 mg, 10-20 mg, 10-30 mg, 10-40 mg, 10-50 mg, 10-70 mg, 10-100 mg, 20-30 mg, 20-40 mg, 20-50 mg, 20-70 mg, 20-100 mg, 30-40 mg, 30-50 mg, 30-70 mg, 30-100 mg, or 40-100 mg. In certain preferred embodiments, COR-001 is administered subcutaneously at a monthly equivalent dose of 3-70 mg.

In various embodiments, COR-001 is administered subcutaneously at a monthly equivalent dose of about 0.5 mg, 1 mg, 2 mg, 3 mg, 4 mg, 5 mg, 6 mg, 7 mg, 8 mg, 9 mg, 10 mg, 20 mg, 30 mg, 40 mg, 50 mg, 70 mg, or 100 mg. In certain embodiments, COR-001 is administered subcutaneously at a monthly equivalent dose of about 1 mg. In certain embodiments, COR-001 is administered subcutaneously at a monthly equivalent dose of about 2 mg. In certain embodiments, COR-001 is administered subcutaneously at a monthly equivalent dose of about 3 mg. In certain embodiments, COR-001 is administered subcutaneously at a monthly equivalent dose of about 4 mg. In certain embodiments, COR-001 is administered subcutaneously at a monthly equivalent dose of about 5 mg. In certain embodiments, COR-001 is administered subcutaneously at a monthly equivalent dose of about 6 mg. In certain embodiments, COR-001 is administered subcutaneously at a monthly equivalent dose of about 10 mg. In certain embodiments, COR-001 is administered subcutaneously at a monthly equivalent dose of about 12 mg. In certain embodiments, COR-001 is administered subcutaneously at a monthly equivalent dose of about 15 mg. In certain embodiments, COR-001 is administered subcutaneously at a monthly equivalent dose of about 17 mg. In certain embodiments, COR-001 is administered subcutaneously at a monthly equivalent dose of about 20 mg. In certain embodiments, COR-001 is administered subcutaneously at a monthly equivalent dose of about 35 mg. In certain embodiments, COR-001 is administered subcutaneously at a monthly equivalent dose of about 40 mg. In certain embodiments, COR-001 is administered subcutaneously at a monthly equivalent dose of about 70 mg. In certain embodiments, COR-001 is administered subcutaneously at a monthly equivalent dose of about 100 mg.

In some embodiments, the IL-6 antagonist is siltuximab. In various embodiments, siltuximab is administered intravenously at a monthly equivalent dose of 10-500 mg, such as 10-20 mg, 10-30 mg, 10-40 mg, 10-50 mg, 10-100 mg, 10-150 mg, 10-200 mg, 10-300 mg, 10-400 mg, 20-30 mg, 20-40 mg, 20-50 mg, 20-100 mg, 20-150 mg, 20-200 mg, 20-300 mg, 20-400 mg, 20-500 mg, 30-40 mg, 30-50 mg, 30-100 mg, 30-150 mg, 30-200 mg, 30-300 mg, 30-400 mg, 30-500 mg, 40-50 mg, 40-100 mg, 40-150 mg, 40-200 mg, 40-250 mg, 40-300 mg, 40-400 mg, 40-500 mg, 50-100 mg, 50-150 mg, 50-200 mg, 50-300 mg, 50-400 mg, 50-500 mg, 100-150 mg, 100-200 mg, 100-300 mg, 100-400 mg, 100-500 mg, 150-200 mg, 150-300 mg, 150-400 mg, 150-500 mg, 200-300 mg, 200-400 mg, 200-500 mg, 300-400 mg, 300-500 mg, or 400-500 mg. In certain preferred embodiments, siltuximab is administered intravenously at a monthly equivalent dose of 50-500 mg. In various embodiments, siltuximab is administered intravenously at a monthly equivalent dose of about 10 mg, 20 mg, 30 mg, 40 mg, 50 mg, 100 mg, 150 mg, 200 mg, 300 mg, 400 mg, or 500 mg. In certain embodiments, siltuximab is administered intravenously at a monthly equivalent dose of about 50 mg. In certain embodiments, siltuximab is administered intravenously at a monthly equivalent dose of about 100 mg. In certain embodiments, siltuximab is administered intravenously at a monthly equivalent dose of about 150 mg. In certain embodiments, siltuximab is administered intravenously at a monthly equivalent dose of about 200 mg. In certain embodiments, siltuximab is administered intravenously at a monthly equivalent dose of about 300 mg. In certain embodiments, siltuximab is administered intravenously at a monthly equivalent dose of about 500 mg.

In various embodiments, siltuximab is administered subcutaneously at a monthly equivalent dose of 50-1000 mg, such as 50-80 mg, 50-100 mg, 50-160 mg, 50-200 mg, 50-240 mg, 50-320 mg, 50-480 mg, 50-800 mg, 80-100 mg, 80-160 mg, 80-200 mg, 80-240 mg, 80-320 mg, 80-480 mg, 80-800 mg, 80-1000 mg, 100-160 mg, 100-200 mg, 100-240 mg, 100-320 mg, 100-480 mg, 100-800 mg, 100-1000 mg, 160-200 mg, 160-240 mg, 160-320 mg, 160-480 mg, 160-800 mg, 160-1000 mg, 200-240 mg, 200-320 mg, 200-480 mg, 200-800 mg, 200-1000 mg, 240-320 mg, 240-480 mg, 240-800 mg, 240-1000 mg, 320-480 mg, 320-800 mg, 320-1000 mg, 480-800 mg, 480-1000 mg, or 800-1000 mg. In certain preferred embodiments, siltuximab is administered subcutaneously at a monthly equivalent dose of 80-800 mg. In various embodiments, siltuximab is administered subcutaneously at a monthly equivalent dose of about 50 mg, 80 mg. 100 mg, 160 mg, 240 mg, 320 mg, 480 mg, 800 mg, or 1000 mg. In certain embodiments, siltuximab is administered subcutaneously at a monthly equivalent dose of about 50 mg. In certain embodiments, siltuximab is administered subcutaneously at a monthly equivalent dose of about 80 mg. In certain embodiments, siltuximab is administered subcutaneously at a monthly equivalent dose of about 100 mg. In certain embodiments, siltuximab is administered subcutaneously at a monthly equivalent dose of about 160 mg. In certain embodiments, siltuximab is administered subcutaneously at a monthly equivalent dose of about 240 mg. In certain embodiments, siltuximab is administered subcutaneously at a monthly equivalent dose of about 320 mg. In certain embodiments, siltuximab is administered subcutaneously at a monthly equivalent dose of about 480 mg. In certain embodiments, siltuximab is administered subcutaneously at a monthly equivalent dose of about 800 mg. In certain embodiments, siltuximab is administered subcutaneously at a monthly equivalent dose of about 1000 mg.

In some embodiments, the IL-6 antagonist is gerilimzumab. In various embodiments, gerilimzumab is administered intravenously at a monthly equivalent dose of 0.05-2 mg, such as 0.05-0.075 mg, 0.05-0.1 mg, 0.05-0.12 mg, 0.05-0.3 mg, 0.05-0.6 mg, 0.05-0.9 mg, 0.05-1.8 mg, 0.075-0.1 mg, 0.075-0.12 mg, 0.075-0.3 mg, 0.075-0.6 mg, 0.075-0.9 mg, 0.075-1.8 mg, 0.075-2 mg, 0.1-0.12 mg, 0.1-0.3 mg, 0.1-0.6 mg, 0.1-0.9 mg, 0.1-1.8 mg, 0.1-2 mg, 0.12-0.3 mg, 0.12-0.6 mg, 0.12-0.9 mg, 0.12-1.8 mg, 0.12-2 mg, 0.3-0.6 mg, 0.3-0.9 mg, 0.3-1.8 mg, 0.3-2 mg, 0.6-0.9 mg, 0.6-1.8 mg, 0.6-2 mg, 0.9-1.8 mg, 0.9-2 mg, or 1.8-2 mg. In certain preferred embodiments, gerilimzumab is administered intravenously at a monthly equivalent dose of 0.075-1.8 mg. In various embodiments, gerilimzumab is administered intravenously at a monthly equivalent dose of about 0.05 mg, 0.075 mg, 0.1 mg, 0.12 mg, 0.3 mg, 0.6 mg, 0.9 mg, 1.8 mg, or 2 mg. In certain embodiments, gerilimzumab is administered intravenously at a monthly equivalent dose of about 0.05 mg. In certain embodiments, gerilimzumab is administered intravenously at a monthly equivalent dose of about 0.075 mg. In certain embodiments, gerilimzumab is administered intravenously at a monthly equivalent dose of about 0.1 mg. In certain embodiments, gerilimzumab is administered intravenously at a monthly equivalent dose of about 0.12 mg. In certain embodiments, gerilimzumab is administered intravenously at a monthly equivalent dose of about 0.3 mg. In certain embodiments, gerilimzumab is administered intravenously at a monthly equivalent dose of about 0.6 mg. In certain embodiments, gerilimzumab is administered intravenously at a monthly equivalent dose of about 0.9 mg. In certain embodiments, gerilimzumab is administered intravenously at a monthly equivalent dose of about 1.8 mg. In certain embodiments, gerilimzumab is administered intravenously at a monthly equivalent dose of about 2 mg.

In various embodiments, gerilimzumab is administered subcutaneously at a monthly equivalent dose of 0.1-5 mg, such as 0.1-0.125 mg, 0.1-0.15 mg, 0.1-0.2 mg, 0.1-0.5 mg, 0.1-1 mg, 0.1-1.5 mg, 0.1-2 mg, 0.1-3 mg, 0.1-4 mg, 0.125-0.15 mg, 0.125-0.2 mg, 0.125-0.5 mg, 0.125-1 mg, 0.125-1.5 mg, 0.125-2 mg, 0.125-3 mg, 0.125-4 mg, 0.125-5 mg, 0.15-0.2 mg, 0.15-0.5 mg, 0.15-1 mg, 0.15-1.5 mg, 0.15-2 mg, 0.15-3 mg, 0.15-4 mg, 0.15-5 mg, 0.2-0.5 mg, 0.2-1 mg, 0.2-1.5 mg, 0.2-2 mg, 0.2-3 mg, 0.2-4 mg, 0.2-5 mg, 0.5-1 mg, 0.5-1.5 mg, 0.5-2 mg, 0.5-3 mg, 0.5-4 mg, 0.5-5 mg, 1-1.5 mg, 1-2 mg, 1-3 mg, 1-4 mg, 1-5 mg, 1.5-2 mg, 1.5-3 mg, 1.5-4 mg, 1.5-5 mg, 2-3 mg, 2-4 mg, 2-5 mg, 3-4 mg, 3-5 mg, or 4-5 mg. In certain preferred embodiments, gerilimzumab is administered subcutaneously at a monthly equivalent dose of 0.125-3 mg. In various embodiments, gerilimzumab is administered subcutaneously at a monthly equivalent dose of about 0.1 mg, 0.125 mg, 0.15 mg, 0.2 mg, 0.5 mg, 1 mg, 1.5 mg, 2 mg, 3 mg, 4 mg, or 5 mg. In certain embodiments, gerilimzumab is administered subcutaneously at a monthly equivalent dose of about 0.125 mg. In certain embodiments, gerilimzumab is administered subcutaneously at a monthly equivalent dose of about 0.2 mg. In certain embodiments, gerilimzumab is administered subcutaneously at a monthly equivalent dose of about 0.5 mg. In certain embodiments, gerilimzumab is administered subcutaneously at a monthly equivalent dose of about 1 mg. In certain embodiments, gerilimzumab is administered subcutaneously at a monthly equivalent dose of about 1.5 mg. In certain embodiments, gerilimzumab is administered subcutaneously at a monthly equivalent dose of about 3 mg.

In some embodiments, the IL-6 antagonist is sirukumab. In various embodiments, sirukumab is administered intravenously at a monthly equivalent dose of 1-80 mg, such as 1-1.5 mg, 1-3 mg, 1-6 mg, 1-12 mg, 1-36 mg, 1-60 mg, 1.5-3 mg, 1.5-6 mg, 1.5-12 mg, 1.5-36 mg, 1.5-60 mg, 1.5-80 mg, 3-6 mg, 3-12 mg, 3-36 mg, 3-60 mg, 3-80 mg, 6-12 mg, 6-36 mg, 6-60 mg, 6-80 mg, 12-36 mg, 12-60 mg, 12-80 mg, 36-60 mg, 36-80 mg, or 60-80 mg. In certain preferred embodiments, sirukumab is administered intravenously at a monthly equivalent dose of 1.5-60 mg. In various embodiments, sirukumab is administered intravenously at a monthly equivalent dose of about 1 mg, 1.5 mg, 3 mg, 6 mg, 12 mg, 36 mg, 60 mg, or 80 mg. In certain embodiments, sirukumab is administered intravenously at a monthly equivalent dose of about 1 mg. In certain embodiments, sirukumab is administered intravenously at a monthly equivalent dose of about 1.5 mg. In certain embodiments, sirukumab is administered intravenously at a monthly equivalent dose of about 3 mg. In certain embodiments, sirukumab is administered intravenously at a monthly equivalent dose of about 6 mg. In certain embodiments, sirukumab is administered intravenously at a monthly equivalent dose of about 12 mg. In certain embodiments, sirukumab is administered intravenously at a monthly equivalent dose of about 36 mg. In certain embodiments, sirukumab is administered intravenously at a monthly equivalent dose of about 60 mg. In certain embodiments, sirukumab is administered intravenously at a monthly equivalent dose of about 80 mg.

In various embodiments, sirukumab is administered subcutaneously at a monthly equivalent dose of 1-100 mg, such as 1-2.5 mg, 1-5 mg, 1-10 mg, 1-20 mg, 1-30 mg, 1-40 mg, 1-50 mg, 1-60 mg, 2.5-5 mg, 2.5-10 mg, 2.5-20 mg, 2.5-30 mg, 2.5-40 mg, 2.5-50 mg, 2.5-60 mg, 2.5-100 mg, 5-10 mg, 5-20 mg, 5-30 mg, 5-40 mg, 5-50 mg, 5-60 mg, 5-100 mg, 10-20 mg, 10-30 mg, 10-40 mg, 10-50 mg, 10-60 mg, 10-100 mg, 20-30 mg, 20-40 mg, 20-50 mg, 20-60 mg, 20-100 mg, 30-40 mg, 30-50 mg, 30-60 mg, 30-100 mg, 40-50 mg, 40-60 mg, 40-100 mg, 50-60 mg, 50-100 mg, or 60-100 mg. In certain preferred embodiments, sirukumab is administered subcutaneously at a monthly equivalent dose of 2.5-100 mg. In various embodiments, sirukumab is administered subcutaneously at a monthly equivalent dose of about 1 mg, 2.5 mg, 5 mg, 10 mg, 20 mg, 30 mg, 40 mg, 50 mg, 60 mg, or 100 mg. In certain embodiments, sirukumab is administered subcutaneously at a monthly equivalent dose of about 2.5 mg. In certain embodiments, sirukumab is administered subcutaneously at a monthly equivalent dose of about 5 mg. In certain embodiments, sirukumab is administered subcutaneously at a monthly equivalent dose of about 10 mg. In certain embodiments, sirukumab is administered subcutaneously at a monthly equivalent dose of about 20 mg. In certain embodiments, sirukumab is administered subcutaneously at a monthly equivalent dose of about 60 mg. In certain embodiments, sirukumab is administered subcutaneously at a monthly equivalent dose of about 100 mg.

In some embodiments, the IL-6 antagonist is clazakizumab. In various embodiments, clazakizumab is administered intravenously at a monthly equivalent dose of 1-80 mg, such as 1-3 mg, 1-6 mg, 1-12 mg, 1-24 mg, 1-36 mg, 1-60 mg, 3-6 mg, 3-12 mg, 3-24 mg, 3-36 mg, 3-60 mg, 3-80 mg, 6-12 mg, 6-24 mg, 6-36 mg, 6-60 mg, 6-80 mg, 12-24 mg, 12-36 mg, 12-60 mg, 12-80 mg, 24-36 mg, 24-60 mg, 24-80 mg, 36-60 mg, 36-80 mg, or 60-80 mg. In certain preferred embodiments, clazakizumab is administered intravenously at a monthly equivalent dose of 3-60 mg. In various embodiments, clazakizumab is administered intravenously at a monthly equivalent dose of about 1 mg, 3 mg, 6 mg, 12 mg, 24 mg, 36 mg, 60 mg or 80 mg. In certain embodiments, clazakizumab is administered intravenously at a monthly equivalent dose of about 1 mg. In certain embodiments, clazakizumab is administered intravenously at a monthly equivalent dose of about 3 mg. In certain embodiments, clazakizumab is administered intravenously at a monthly equivalent dose of about 6 mg. In certain embodiments, clazakizumab is administered intravenously at a monthly equivalent dose of about 12 mg. In certain embodiments, clazakizumab is administered intravenously at a monthly equivalent dose of about 24 mg. In certain embodiments, clazakizumab is administered intravenously at a monthly equivalent dose of about 36 mg. In certain embodiments, clazakizumab is administered intravenously at a monthly equivalent dose of about 60 mg. In certain embodiments, clazakizumab is administered intravenously at a monthly equivalent dose of about 80 mg.

In various embodiments, clazakizumab is administered subcutaneously at a monthly equivalent dose of 1-100 mg, such as 1-2 mg, 1-5 mg, 1-10 mg, 1-20 mg, 1-30 mg, 1-40 mg, 1-50 mg, 1-60 mg, 2-5 mg, 2-10 mg, 2-20 mg, 2-30 mg, 2-40 mg, 2-50 mg, 2-60 mg, 2-100 mg, 5-10 mg, 5-20 mg, 5-30 mg, 5-40 mg, 5-50 mg, 5-60 mg, 5-100 mg, 10-20 mg, 10-30 mg, 10-40 mg, 10-50 mg, 10-60 mg, 10-100 mg, 20-30 mg, 20-40 mg, 20-50 mg, 20-60 mg, 20-100 mg, 30-40 mg, 30-50 mg, 30-60 mg, 30-100 mg, 40-50 mg, 40-60 mg, 40-100 mg, 50-60 mg, 50-100 mg, or 60-100 mg. In certain preferred embodiments, clazakizumab is administered subcutaneously at a monthly equivalent dose of 5-100 mg. In various embodiments, clazakizumab is administered subcutaneously at a monthly equivalent dose of about 1 mg, 2 mg, 5 mg, 10 mg, 20 mg, 30 mg, 40 mg, 50 mg, 60 mg, or 100 mg. In certain embodiments, clazakizumab is administered subcutaneously at a monthly equivalent dose of about 5 mg. In certain embodiments, clazakizumab is administered subcutaneously at a monthly equivalent dose of about 10 mg. In certain embodiments, clazakizumab is administered subcutaneously at a monthly equivalent dose of about 20 mg. In certain embodiments, clazakizumab is administered subcutaneously at a monthly equivalent dose of about 40 mg. In certain embodiments, clazakizumab is administered subcutaneously at a monthly equivalent dose of about 60 mg. In certain embodiments, clazakizumab is administered subcutaneously at a monthly equivalent dose of about 100 mg.

In some embodiments, the IL-6 antagonist is olokizumab. In various embodiments, olokizumab is administered intravenously at a monthly equivalent dose of 1-80 mg, such as 1-1.8 mg, 1-3.6 mg, 1-9 mg, 1-18 mg, 1-45 mg, 1-60 mg, 1.8-3.6 mg, 1.8-9 mg, 1.8-18 mg, 1.8-45 mg, 1.8-60 mg, 1.8-80 mg, 3.6-9 mg, 3.6-18 mg, 3.6-45 mg, 3.6-60 mg, 3.6-80 mg, 9-18 mg, 9-45 mg, 9-60 mg, 9-80 mg, 18-45 mg, 18-60 mg, 18-80 mg, 45-60 mg, 45-80 mg, or 60-80 mg. In certain preferred embodiments, olokizumab is administered intravenously at a monthly equivalent dose of 1.8-60 mg. In various embodiments, olokizumab is administered intravenously at a monthly equivalent dose of about 1 mg, 1.8 mg, 3.6 mg, 9 mg, 18 mg, 45 mg, 60 mg, or 80 mg. In certain embodiments, olokizumab is administered intravenously at a monthly equivalent dose of about 1 mg. In certain embodiments, olokizumab is administered intravenously at a monthly equivalent dose of about 1.8 mg. In certain embodiments, olokizumab is administered intravenously at a monthly equivalent dose of about 3.6 mg. In certain embodiments, olokizumab is administered intravenously at a monthly equivalent dose of about 9 mg. In certain embodiments, olokizumab is administered intravenously at a monthly equivalent dose of about 18 mg. In certain embodiments, olokizumab is administered intravenously at a monthly equivalent dose of about 45 mg. In certain embodiments, olokizumab is administered intravenously at a monthly equivalent dose of about 60 mg. In certain embodiments, olokizumab is administered intravenously at a monthly equivalent dose of about 80 mg.

In various embodiments, olokizumab is administered subcutaneously at a monthly equivalent dose of 1-100 mg, such as 1-3 mg, 1-6 mg, 1-10 mg, 1-15 mg, 1-20 mg, 1-30 mg, 1-50 mg, 1-72 mg, 3-6 mg, 3-10 mg, 3-15 mg, 3-20 mg, 3-30 mg, 3-50 mg, 3-72 mg, 3-100 mg, 6-10 mg, 6-15 mg, 6-20 mg, 6-30 mg, 6-50 mg, 6-72 mg, 6-100 mg, 10-15 mg, 10-20 mg, 10-30 mg, 10-50 mg, 10-72 mg, 10-100 mg, 15-20 mg, 15-30 mg, 15-50 mg, 15-72 mg, 15-100 mg, 20-30 mg, 20-50 mg, 20-72 mg, 20-100 mg, 30-50 mg, 30-72 mg, 30-100 mg, 50-72 mg, 50-100 mg, or 72-100 mg. In certain preferred embodiments, olokizumab is administered subcutaneously at a monthly equivalent dose of 3-100 mg. In various embodiments, olokizumab is administered subcutaneously at a monthly equivalent dose of about 1 mg, 3 mg, 6 mg, 10 mg, 15 mg, 20 mg, 30 mg, 50 mg, 72 mg, or 100 mg. In certain embodiments, olokizumab is administered subcutaneously at a monthly equivalent dose of about 3 mg. In certain embodiments, olokizumab is administered subcutaneously at a monthly equivalent dose of about 6 mg. In certain embodiments, olokizumab is administered subcutaneously at a monthly equivalent dose of about 15 mg. In certain embodiments, olokizumab is administered subcutaneously at a monthly equivalent dose of about 30 mg. In certain embodiments, olokizumab is administered subcutaneously at a monthly equivalent dose of about 72 mg. In certain embodiments, olokizumab is administered subcutaneously at a monthly equivalent dose of about 100 mg.

In some embodiments, the IL-6 antagonist is tocilizumab. In various embodiments, tocilizumab is administered intravenously at a monthly equivalent dose of 10-500 mg, such as 10-20 mg, 10-50 mg, 10-100 mg, 10-150 mg, 10-200 mg, 10-250 mg, 10-300 mg, 10-350 mg, 10-400 mg, 20-50 mg, 20-100 mg, 20-150 mg, 20-200 mg, 20-250 mg, 20-300 mg, 20-350 mg, 20-400 mg, 20-500 mg, 50-100 mg, 50-150 mg, 50-200 mg, 50-250 mg, 50-300 mg, 50-350 mg, 50-400 mg, 50-500 mg, 100-150 mg, 100-200 mg, 100-250 mg, 100-300 mg, 100-350 mg, 100-400 mg, 100-500 mg, 150-200 mg, 150-250 mg, 150-300 mg, 150-350 mg, 150-400 mg, 150-500 mg, 200-250 mg, 200-300 mg, 200-350 mg, 200-400 mg, 200-500 mg, 250-300 mg, 250-350 mg, 250-400 mg, 250-500 mg, 300-350 mg, 300-400 mg, 300-500 mg, 350-400 mg, 350-500 mg, or 400-500 mg. In certain preferred embodiments, tocilizumab is administered intravenously at a monthly equivalent dose of 50-500 mg. In various embodiments, tocilizumab is administered intravenously at a monthly equivalent dose of about 10 mg, 20 mg, 30 mg, 40 mg, 50 mg, 100 mg, 150 mg, 200 mg, 250 mg, 300 mg, 350 mg, 400 mg, or 500 mg. In certain embodiments, tocilizumab is administered intravenously at a monthly equivalent dose of about 50 mg. In certain embodiments, tocilizumab is administered intravenously at a monthly equivalent dose of about 100 mg. In certain embodiments, tocilizumab is administered intravenously at a monthly equivalent dose of about 150 mg. In certain embodiments, tocilizumab is administered intravenously at a monthly equivalent dose of about 250 mg. In certain embodiments, tocilizumab is administered intravenously at a monthly equivalent dose of about 350 mg. In certain embodiments, tocilizumab is administered intravenously at a monthly equivalent dose of about 500 mg.

In various embodiments, tocilizumab is administered subcutaneously at a monthly equivalent dose of 50-1000 mg, such 50-80 mg, 50-160 mg, 50-240 mg, 50-400 mg, 50-560 mg, 50-800 mg, 80-160 mg, 80-240 mg, 80-400 mg, 80-560 mg, 80-800 mg, 80-1000 mg, 160-240 mg, 160-400 mg, 160-560, 160-800 mg, 160-1000 mg, 240-400 mg, 240-560 mg, 240-800 mg, 240-1000 mg, 400-560 mg, 400-800 mg, 400-1000 mg, 560-800 mg, 560-1000 mg, or 800-100 mg. In certain preferred embodiments, tocilizumab is administered subcutaneously at a monthly equivalent dose of 80-800 mg. In various embodiments, tocilizumab is administered subcutaneously at a monthly equivalent dose of about 50 mg, 80 mg, 160 mg, 240 mg, 400 mg, 560 mg, 800 mg, or 1000 mg. In certain embodiments, tocilizumab is administered subcutaneously at a monthly equivalent dose of about 50 mg. In certain embodiments, tocilizumab is administered subcutaneously at a monthly equivalent dose of about 80 mg. In certain embodiments, tocilizumab is administered subcutaneously at a monthly equivalent dose of about 160 mg. In certain embodiments, tocilizumab is administered subcutaneously at a monthly equivalent dose of about 240 mg. In certain embodiments, tocilizumab is administered subcutaneously at a monthly equivalent dose of about 400 mg. In certain embodiments, tocilizumab is administered subcutaneously at a monthly equivalent dose of about 560 mg. In certain embodiments, tocilizumab is administered subcutaneously at a monthly equivalent dose of about 800 mg. In certain embodiments, tocilizumab is administered subcutaneously at a monthly equivalent dose of about 1000 mg.

In some embodiments, the IL-6 antagonist is sarilumab. In various embodiments, sarilumab is administered intravenously at a monthly equivalent dose of 10-150 mg, such as 10-12 mg, 10-24 mg, 10-48 mg, 10-60 mg, 10-72 mg, 10-120 mg, 12-24 mg, 12-48 mg, 12-60 mg, 12-72 mg, 12-120 mg, 12-150 mg, 24-48 mg, 24-60 mg, 24-72 mg, 24-120 mg, 24-150 mg, 48-60 mg, 48-72 mg, 48-120 mg, 48-150 mg, 60-72 mg, 60-120 mg, 60-150 mg, 72-120 mg, 72-150 mg, or 120-150 mg. In certain preferred embodiments, sarilumab is administered intravenously at a monthly equivalent dose of 12-120 mg. In various embodiments, sarilumab is administered intravenously at a monthly equivalent dose of 10 mg, 12 mg, 24 mg, 48 mg, 60 mg, 72 mg, 120 mg, or 150 mg. In certain embodiments, sarilumab is administered intravenously at a monthly equivalent dose of 10 mg. In certain embodiments, sarilumab is administered intravenously at a monthly equivalent dose of 12 mg. In certain embodiments, sarilumab is administered intravenously at a monthly equivalent dose of 24 mg. In certain embodiments, sarilumab is administered intravenously at a monthly equivalent dose of 48 mg. In certain embodiments, sarilumab is administered intravenously at a monthly equivalent dose of 60 mg. In certain embodiments, sarilumab is administered intravenously at a monthly equivalent dose of 72 mg. In certain embodiments, sarilumab is administered intravenously at a monthly equivalent dose of 120 mg. In certain embodiments, sarilumab is administered intravenously at a monthly equivalent dose of 150 mg.

In various embodiments, sarilumab is administered subcutaneously at a monthly equivalent dose of 10-200 mg, such as 10-20 mg, 10-40 mg, 10-60 mg, 10-80 mg, 10-100 mg, 10-120 mg, 20-40 mg, 20-60 mg, 20-80 mg, 20-100 mg, 20-120 mg, 20-200 mg, 40-60 mg, 40-80 mg, 40-100 mg, 40-120 mg, 40-200 mg, 60-80 mg, 60-100 mg, 60-120 mg, mg, 60-200 mg, 80-100 mg, 80-120 mg, 80-200 mg, 100-120 mg, 100-200 mg, or 120-200 mg. In certain preferred embodiments, sarilumab is administered subcutaneously at a monthly equivalent dose of 20-200 mg. In various embodiments, sarilumab is administered subcutaneously at a monthly equivalent dose of about 10 mg, 20 mg, 30 mg, 40 mg, 50 mg, 60 mg, 80 mg, 100 mg, 120 mg, 150 mg, or 200 mg. In certain embodiments, sarilumab is administered subcutaneously at a monthly equivalent dose of about 20 mg. In certain embodiments, sarilumab is administered subcutaneously at a monthly equivalent dose of about 40 mg. In certain embodiments, sarilumab is administered subcutaneously at a monthly equivalent dose of about 80 mg. In certain embodiments, sarilumab is administered subcutaneously at a monthly equivalent dose of about 100 mg. In certain embodiments, sarilumab is administered subcutaneously at a monthly equivalent dose of about 120 mg. In certain embodiments, sarilumab is administered subcutaneously at a monthly equivalent dose of about 200 mg.

In some embodiments, the IL-6 antagonist is vobarilizumab. In various embodiments, vobarilizumab is administered intravenously at a monthly equivalent dose of 2-150 mg, such as 2-4 mg, 2-6 mg, 2-30 mg, 2-60 mg, 2-84 mg, 2-120 mg, 4-6 mg, 4-30 mg, 4-60 mg, 4-84 mg, 4-120 mg, 4-150 mg, 6-30 mg, 6-60 mg, 6-84 mg, 6-120 mg, 6-150 mg, 30-60 mg, 30-84 mg, 30-120 mg, 30-150 mg, 60-84 mg, 60-120 mg, 60-150 mg, 84-120 mg, 84-150 mg, or 120-150 mg. In certain preferred embodiments, vobarilizumab is administered intravenously at a monthly equivalent dose of 4-120 mg. In various embodiments, vobarilizumab is administered intravenously at a monthly equivalent dose of about 2 mg, 4 mg, 6 mg, 30 mg, 60 mg, 84 mg, 120 mg, or 150 mg. In certain embodiments, vobarilizumab is administered intravenously at a monthly equivalent dose of about 2 mg. In certain embodiments, vobarilizumab is administered intravenously at a monthly equivalent dose of about 4 mg. In certain embodiments, vobarilizumab is administered intravenously at a monthly equivalent dose of about 6 mg. In certain embodiments, vobarilizumab is administered intravenously at a monthly equivalent dose of about 30 mg. In certain embodiments, vobarilizumab is administered intravenously at a monthly equivalent dose of about 60 mg. In certain embodiments, vobarilizumab is administered intravenously at a monthly equivalent dose of about 84 mg. In certain embodiments, vobarilizumab is administered intravenously at a monthly equivalent dose of about 120 mg. In certain embodiments, vobarilizumab is administered intravenously at a monthly equivalent dose of about 150 mg.

In various embodiments, vobarilizumab is administered subcutaneously at a monthly equivalent dose of 5-200 mg, such as 5-7 mg, 5-10 mg, 5-20 mg, 5-50 mg, 5-70 mg, 5-100 mg, 5-140 mg, 7-10 mg, 7-20 mg, 7-50 mg, 7-70 mg, 7-100 mg, 7-140 mg, 7-200 mg, 10-20 mg, 10-50 mg, 10-70 mg, 10-100 mg, 10-140 mg, 10-200 mg, 20-50 mg, 20-70 mg, 20-100 mg, 20-140 mg, 20-200 mg, 50-70 mg, 50-100 mg, 50-140 mg, 50-200 mg, 70-100 mg, 70-140 mg, 70-200 mg, 100-140 mg, 100-200 mg, or 140-200 mg. In certain preferred embodiments, vobarilizumab is administered subcutaneously at a monthly equivalent dose of 7-200 mg. In various embodiments, vobarilizumab is administered subcutaneously at a monthly equivalent dose of about 5 mg, 7 mg, 10 mg, 20 mg, 40 mg, 50 mg, 70 mg, 100 mg, 140 mg, or 200 mg. In certain embodiments, vobarilizumab is administered subcutaneously at a monthly equivalent dose of about 7 mg. In certain embodiments, vobarilizumab is administered subcutaneously at a monthly equivalent dose of about 10 mg. In certain embodiments, vobarilizumab is administered subcutaneously at a monthly equivalent dose of about 50 mg. In certain embodiments, vobarilizumab is administered subcutaneously at a monthly equivalent dose of about 100 mg. In certain embodiments, vobarilizumab is administered subcutaneously at a monthly equivalent dose of about 140 mg. In certain embodiments, vobarilizumab is administered subcutaneously at a monthly equivalent dose of about 200 mg.

### 3. Examples

The following examples are provided by way of exemplification and illustration, not limitation.

### 3.1. Example 1: Phase 1/2 Clinical Study

A Phase 1/2 clinical study was conducted to assess the safety, pharmacokinetics, and pharmacodynamics of multiple IV doses of COR-001.

### 3.1.1. Drug Product (COR-001)

COR-001 is a human IgG1, kappa antibody directed against interleukin-6 (IL-6). COR-001 contains a “YTE” mutation in its Fc region. The sequence and other features of COR-001 are described in Chapter 2.7.1.1 above.

### 3.1.2. Study Design

The study was a randomized, double-blind, placebo-controlled trial designed to evaluate the safety, pharmacokinetics, and pharmacodynamic effects of multiple doses of COR-001 (MEDI5117) or placebo administered to sequential cohorts of hemodialysis patients.

Key inclusion criteria include stage 5 chronic kidney disease (CKD-5) on hemodialysis, positive for TMPRSS6 736A genotype (major allele), IL-6 level greater than 4 pg/mL, and erythropoietic resistive index greater than 8.

Ten hemodialysis patients were randomized to COR-001 or placebo within each dosing cohort. When a higher dose than studied in a prior cohort was initiated, the first 2 (sentinel) patients in that cohort (randomized 1:1 to COR-001 or placebo) were randomized first and the remaining patients were randomized at least 48 hours later, in a 7:1 ratio of COR-001 to placebo. The final ratio of patients treated with COR-001 vs. placebo were 8:2 in each cohort of 10 patients. The maximum tolerated dose (MTD) assessment was based on safety data from Weeks 1 to 3. If more than 2 of 8 active patients in a cohort experienced a dose-limiting toxicity (DLT), the MTD was considered to have been exceeded.

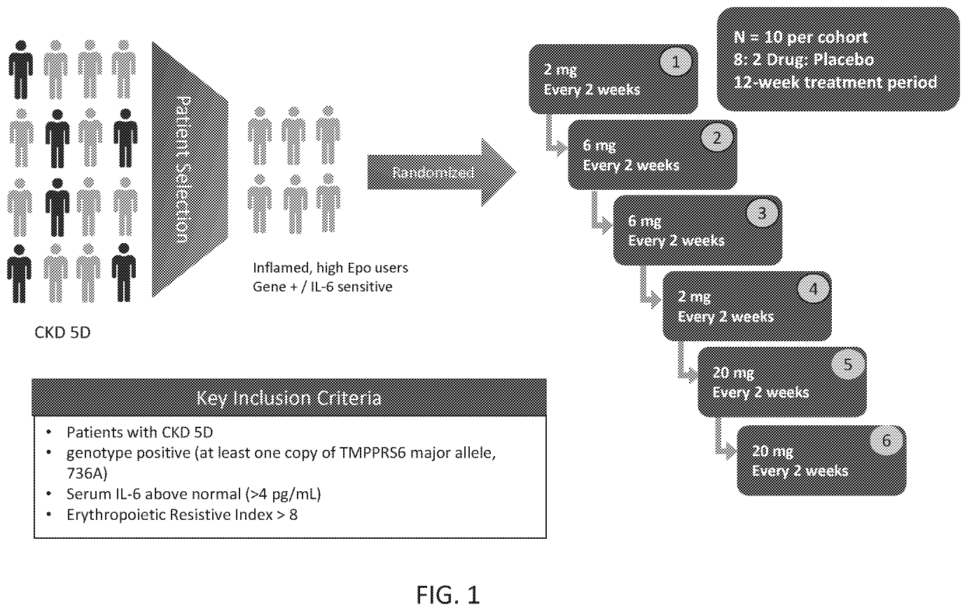

The Dose Escalation Schematic is shown in FIG. 1. COR-001 was administered as an intravenous infusion, started any time before the last 1 hour of the dialysis treatment. The COR-001 dose regimens are shown in Table 1 below.

TABLE 1

| Dose | | Number of | Total Cumulative |
|---|---|---|---|
| Cohort | Dose Regimen | Doses | Dose |
| 1 | 2 mg every 14 days | 6 | 12 mg |
| 2 | 6 mg every 14 days | 6 | 36 mg |
| 3 | 6 mg every 14 days | 6 | 36 mg |
| 4 | 2 mg every 14 days | 6 | 12 mg |
| 5 | 20 mg every 14 days | 6 | 120 mg |
| 6 | 20 mg every 14 days | 6 | 120 mg |

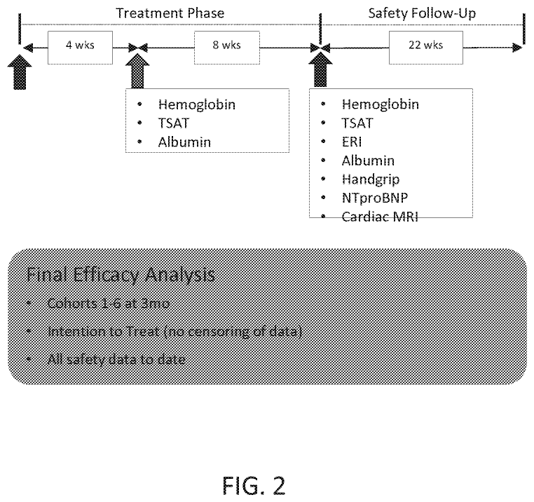

The total study duration for an individual patient was approximately 9 months, excluding the screening period of up to 4 weeks. As shown in FIG. 2, the study included a treatment period of 12 weeks (Week 1 through Week 12), a safety follow-up period of 12 weeks (Week 13 through Week 24), and an extended follow-up period of 10 weeks (Week 25 through Week 35).

Interim study-collected data were summarized by treatment group for the appropriate analysis population, using descriptive statistics. Descriptive statistics for continuous variables included number of patients (n), mean, standard deviation (SD), median, quartiles (Q1 and Q3), minimum (min) and maximum (max) values. Analysis of categorical variables included frequency and percentages.

The changes in high-sensitivity C-reactive protein (hsCRP), absolute neutrophil count (ANC), lipoprotein(a) level, LDL level, hemoglobin, transferrin saturation (TSAT), albumin, erythropoietic resistive index (ERI), handgrip, NT-proBNP, and cardiac MRI were recorded during the study.

### 3.1.3. Analysis of Clinical Data

Analyses were performed to determine the effect of COR-001 on C-reactive protein (CRP), the effect of COR-001 on hemoglobin level, the effect of COR-001 on various cardiac parameters, and the effect of COR-001 on levels of neutrophils and platelets.

C-reactive protein (CRP) is a marker of inflammation. CRP levels increase in response to inflammation, and can be measured with an hsCRP (high-sensitivity C-reactive protein) test. The hsCRP level was measured over the course of the treatment period and the safety follow-up period in patients of the placebo-treated, 2 mg dose regimen, 6 mg dose regimen, and 20 mg dose regimen groups, respectively.

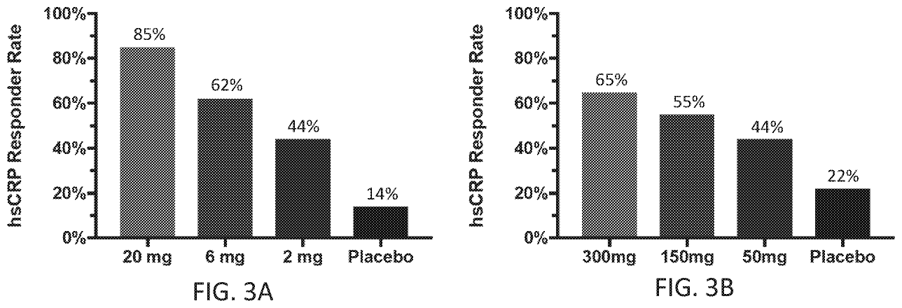

The percentages of patients with post-treatment average hsCRP<2 mg/L at Week 12 were 44%, 62%, and 85% in the 2 mg dose regimen, 6 mg dose regimen, and 20 mg dose regimen groups, respectively, as compared to 14% in the placebo group. The hsCRP responder analysis shows that COR-001 (anti-IL-6) has a superior effect on hsCRP than has been reported for canakinumab (anti-IL1β) in the CANTOS trial. The hsCRP responder rates of COR-001 in stage 5 chronic kidney disease patients on dialysis at IV doses of 20 mg and 6 mg (FIG. 3A) were higher than the hsCRP responder rates of canakinumab at equivalent doses in the CANTOS trial (FIG. 3B). The in vivo IC50 concentration of COR-001 for CRP (50% reduction of baseline CRP) is 206 ng/mL.

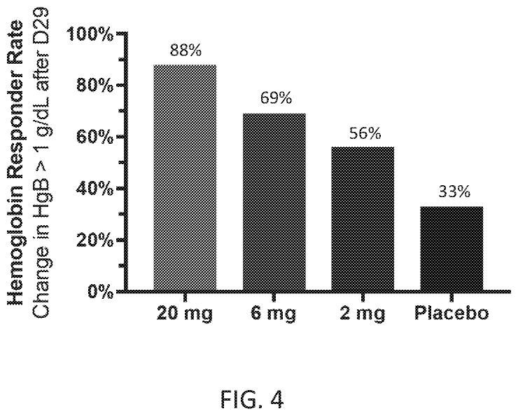

COR-001 improved a primary indicator of anemia, hemoglobin levels. The hemoglobin responder analysis indicated a dose-dependent hemoglobin responder rate of COR-001 treatment (FIG. 4).

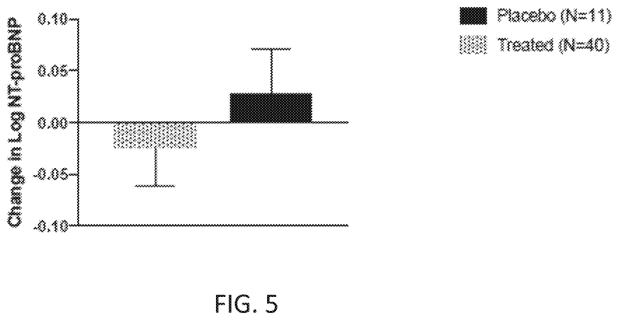

The effect of COR-001 on various biomarkers of heart failure was determined. As shown in FIG. 5, COR-001 decreased the level of the N-terminal prohormone of brain natriuretic peptide (NT-proBNP). The result indicates that treatment of COR-001 can reduce heart failure.

Anti-inflammatory therapies in general, and IL-6 inhibitory therapies in particular, create a risk of inducing immune suppression, thereby promoting the emergence of infections, sometimes serious in nature. Immune suppression can be measured by neutrophil counts. The effect of COR-001 on neutrophil counts was determined.

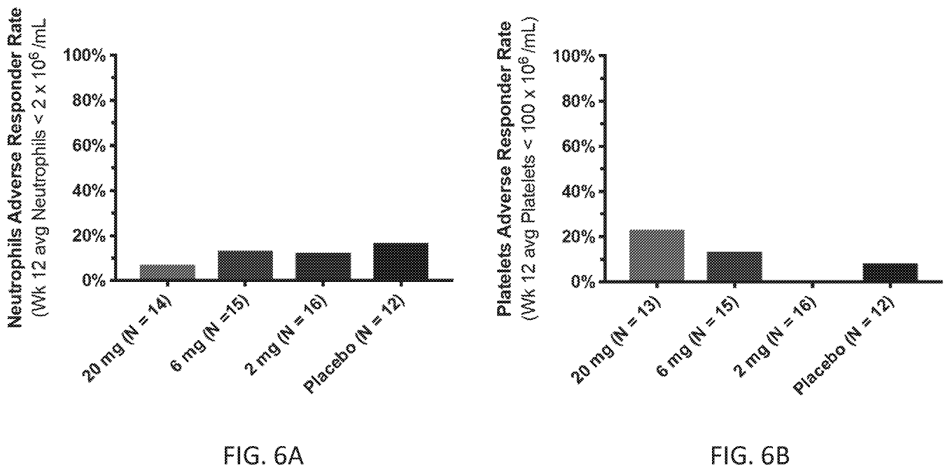

Surprisingly, despite significant reduction in inflammation, as measured by hsCRP levels (FIG. 3A), the absolute neutrophil count of patients treated with COR-001 did not decline to below normal levels. No opportunistic infections were observed during the treatment. As shown in FIG. 6A, the percentages of patients with absolute neutrophil count below 2.0×10⁹/L were not increased with COR-001 at all tested doses as compared to the placebo group. All patients treated with COR-001 at all tested doses had an absolute neutrophil count above 1.5×10⁹/L. The in vivo IC50 concentration of COR-001 for neutrophil count (50% reduction of baseline neutrophil count) is 5540 ng/ml.

The percentages of patients with platelet count below 100×10⁹/L were less than 30% with COR-001 for all tested doses (FIG. 6B). The in vivo IC50 concentration of COR-001 for platelet count (50% reduction of baseline platelet count) is 13800 ng/mL.

In summary, the clinical data indicate that COR-001 treatment at doses of 2 mg, 6 mg, and 20 mg can reduce inflammation without inducing immune suppression in patients with stage 5 chronic kidney disease (CKD-5) on dialysis, whereas the absolute neutrophil count was not decreased significantly in patients treated with COR-001.

Administration of COR-001 reduced CRP in a dose-dependent matter. In addition, COR-001 increased hemoglobin level in these patients. COR-001 decreased the biomarkers of heart failure NT-proBNP.

### 4. Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

### 5. Equivalents

While various specific embodiments have been illustrated and described, the above specification is not restrictive. It will be appreciated that various changes can be made without departing from the spirit and scope of the invention(s). Many variations will become apparent to those skilled in the art upon review of this specification.

1.-24. (canceled)

25. A method of treating a patient who has atherosclerotic cardiovascular disease with inflammation, wherein the patient has KDOQI stage 3-5 chronic kidney disease (CKD), comprising:
administering a dose of 5-20 mg of COR-001 subcutaneously once monthly.

26. The method of claim 25, wherein the dose is 15 mg.

27. The method of claim 25, wherein the administration is sufficient to reduce nonfatal myocardial infarction, nonfatal stroke, and/or cardiovascular death.

28. The method of claim 25, wherein the patient has a C-Reactive Protein (CRP) level greater than 2 mg/L.

29. The method of claim 28, wherein the dose is sufficient to reduce the CRP level to 2 mg/L or less.

30. The method of claim 25, wherein the dose does not cause a decrease in absolute neutrophil count (ANC) below 1500 cells/μL.

31. The method of claim 25, wherein post-treatment low-density lipoprotein (LDL) level is increased by no more than 10% as compared to pre-treatment levels.

32. The method of claim 25, wherein the administration reduces the risk of heart failure and/or cardiovascular death.

33. The method of claim 25, wherein the administration increases cardiac function.

34. The method of claim 25, wherein the administration reduces fibrosis after acute myocardial infarction.

35. A method of treating a patient who has atherosclerotic cardiovascular disease with inflammation, wherein the patient has KDOQI stage 3-5 chronic kidney disease (CKD), comprising:
administering COR-001 subcutaneously at a monthly equivalent dose of 5-20 mg.

36. The method of claim 35, wherein the dose is 15 mg.

37. The method of claim 35, wherein the administration is sufficient to reduce nonfatal myocardial infarction, nonfatal stroke, and/or cardiovascular death.

38. The method of claim 35, wherein the patient has a C-Reactive Protein (CRP) level greater than 2 mg/L.

39. The method of claim 38, wherein the dose is sufficient to reduce the CRP level to 2 mg/L or less.

40. The method of claim 35, wherein the dose does not cause a decrease in absolute neutrophil count (ANC) below 1500 cells/μL.

41. The method of claim 35, wherein post-treatment low-density lipoprotein (LDL) level is increased by no more than 10% as compared to pre-treatment levels.

42. The method of claim 35, wherein the administration reduces the risk of heart failure and/or cardiovascular death.

43. The method of claim 35, wherein the administration increases cardiac function.

44. The method of claim 35, wherein the administration reduces fibrosis after acute myocardial infarction.


SEQUENCE LISTING (14 sequences)

SEQ ID NO: 1 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 2 - DNA, 1201 bp, Homo sapiens
  source 1..1201: mol_type = genomic DNA
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 3 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 4 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 5 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 6 - protein, 84 aa, Homo sapiens
MALSSQIWAACLLLLLLLASLTSGSVFPQQTGQLAELQPQDRAGARASWMPMFQRRRRRDTHFPICIFCCGCCHRSKCGMCCKT

SEQ ID NO: 7 - protein, 5 aa, synthetic construct
  REGION 1..5: note = Description of Artificial Sequence: Synthetic peptide
SNYMI

SEQ ID NO: 8 - protein, 17 aa, synthetic construct
  REGION 1..17: note = Description of Artificial Sequence: Synthetic peptide
DLYYYAGDTYYADSVKG

SEQ ID NO: 9 - protein, 11 aa, synthetic construct
  REGION 1..11: note = Description of Artificial Sequence: Synthetic peptide
WADDHPPWIDL

SEQ ID NO: 10 - protein, 11 aa, synthetic construct
  REGION 1..11: note = Description of Artificial Sequence: Synthetic peptide
RASQGISSWLA

SEQ ID NO: 11 - protein, 7 aa, synthetic construct
  REGION 1..7: note = Description of Artificial Sequence: Synthetic peptide
KASTLES

SEQ ID NO: 12 - protein, 8 aa, synthetic construct
  REGION 1..8: note = Description of Artificial Sequence: Synthetic peptide
QQSWLGGS

SEQ ID NO: 13 - protein, 450 aa, synthetic construct
  REGION 1..450: note = Description of Artificial Sequence: Synthetic polypeptide
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 213 aa, synthetic construct
  REGION 1..213: note = Description of Artificial Sequence: Synthetic polypeptide
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC